(19) United States
(12) Patent Application Publication
(10) Pub. No.: US 2010/0184212 A1
(43) Pub. Date: Jul. 22, 2010

(54) HUMAN EMBRYONIC STEM CELL DERIVED MESODERM-LIKE EPITHELIUM TRANSITIONS TO MESENCHYMAL PROGENITOR CELLS

(76) Inventors: Steven Stice, Athens, GA (US); Nolan Boyd, Louisville, KY (US)

Correspondence Address: COLEMAN SUDOL SAPONE, P.C., 714 COLORADO AVENUE, BRIDGE PORT, CT, 06605-1601, US

(21) Appl. No.: 12/451,720

(22) PCT Filed: May 30, 2008
(86) PCT No.: PCT/US2008/006920

§ 371 Date: Mar. 19, 2010

Related U.S. Application Data
(60) Provisional application No. 60/932,328, filed on May 30, 2007.

(51) Int. Cl. C12N 5/0735; C12N 5/0789; C12N 5/077

(52) U.S. Cl. 435/366; 435/377; 435/325

(57) ABSTRACT

Human embryonic stem cells (hESC) have the potential to produce all of the cells in the body. They are also able to self-renew indefinitely, sparking the hope they could be used as a source for large scale production of therapeutic cell lines. The present invention relates to a monolayer differentiation culture system that induces hESC (WA09 and BG01) to form epithelial sheets with mesodermal gene expression patterns (BMP4, RUNX1, GAT A4). These E-cadherin+ CD90lovv cells then undergo apparent epithelial-mesenchymal transformation (EMT) for the derivation of mesenchymal progenitor cells (hES-MC) that by flow cytometry are negative for hematopoietic (CD34, CD45 and CD 133) and endothelial (CD31 and CD 146) markers, but positive for markers associated with mesenchymal stem cells (MSC) (CD73, CD90, CD105 and CD166). To determine their functionality, we tested their capacity to produce the three lineages commonly associated with MSC and found they could form osteogenic and chondrogenic, but not adipogenic lineages. The derived hES-MC were able to remodel and contract collagen I lattice constructs to an equivalent degree as keloid fibroblast control cells and were induced to express αSMA when exposed to TGF-β1, but not PDGF-B. This data suggests the derived hES-MC cells are multipotent cells with potential uses in tissue engineering/regenerative medicine and for providing a highly reproducible cell source for adult-like progenitor cells.

### RELATED APPLICATIONS, CLAIM OF PRIORITY AND GRANT SUPPORT

The present application claims priority from U.S. provisional application No. 60/932,328, entitled “BMP4 Networks and hES-MSC Cultures”, filed May 30, 2007, the entire contents of which is incorporated by reference in its entirety herein.

The subject matter of the present application was supported by a grant from the National Science Foundation, grant no. NSF EEC-9731643. Consequently, the government has retained certain rights in the invention.

### FIELD OF THE INVENTION

The present invention relates to a unique embryonic stem cell (hESC) culture system for the derivation of mesenchymal stem cells (hMSC) or a hMSC-like cell. In a preferred embodiment, hESC (e.g., BG01, WA09) are grown as a monolayer on laminin coated dishes in microvascular endothelial growth medium 2 (EGM-2-MV) for 30 days. The monolayer differentiation culture system induces hESC (WA09 and BG01) to form epithelial sheets with mesodermal gene expression patterns (BMP4, RUNX1, GATA4). These E-cadherin⁺CD90^{low} cells then undergo apparent epithelial-mesenchymal transformation (EMT) for the derivation of mesenchymal progenitor cells (hES-MC) that by flow cytometry are negative for hematopoietic (CD34, CD45 and CD133) and endothelial (CD31 and CD146) markers, but positive for markers associated with mesenchymal stem cells (MSC) (CD73, CD90, CD105 and CD166). This culture system could be used to produce multipotential cells from hESC with the capacity to recapitulate the differentiation capacity of MSC derived from adult sources. These cells may be used as a cell source for regenerative medicine and tissue engineering as well as prove to be valuable for cell based assays for disease research and drug screening.

### BACKGROUND OF THE INVENTION

The potential of embryonic stem cells to produce all the cells of the body has been proven by producing chimeric mice and noting the body wide contribution of the introduced stem cells [1]. In vitro, differentiating stem cells form embryoid bodies capable of producing all three germ layers [2-4] illustrating the utility of embryonic stem cells as in vitro models of early development. Epithelial-mesenchymal transition (EMT) [5] is the morphological change from the epithelial cell-cell contact to the migratory mesenchymal cell-matrix phenotype. This progression is required for multiple developmental events including gastrulation [6], neural crest delamination [7], coronary vasculature [8] heart valve formation [9] and malignant tumor metastasis [10,11]. There is evidence EMT can be modeled by stem cells [12-14].

With the isolation of human embryonic stem cells (hESC) [15] there is the potential to direct their differentiation toward specific lineages for large scale production in therapeutic applications. Another source of cells are mesenchymal stem cells (MSC) typically isolated from the bone marrow of adults [16]. These cells are multipotent being able to differentiate along osteogenic, chondrogenic and adipogenic lineages [16,17]. Although believed not to be as plastic and limited in their proliferation compared to embryonic stem cells, major advantages to their use are ease of culture, karyotype stability and lack of tumor formation in vivo [18]. Several groups have reported producing MSC-like cells from hESC by multiple methods that include culture on OP9 feeders (stromal cells isolated from op/op calvaria), manual selection of differentiating cells in hESC colonies and sorting on common MSC markers (CD73 or CD105) [19-22] indicating hESC can produce cells similar or equivalent to adult MSC.

### BRIEF DESCRIPTION OF THE FIGURES

FIG. 1 shows hESC monolayer differentiation. hESC were differentiated in EGM2-MV for 20-30 d. A) Within 5 d epithelium appeared (arrow), B) expanded in a circular pattern (arrow) until C) the entire culture presented an epithelial phenotype. D) The epithelial phenotype under went EMT with passaging. E) Time line for differentiation of hESC to epithelium and EMT. 10×

FIG. 2 shows hESC derived epithelial cells express mesodermal markers. RNA was acquired on d0, 5, 10, 15, 20, 25 and 30 and examined by qRT-PCR with respect to 18 S, normalized to d0 and the transformed data [ln(RQ)] analyzed for significance (*p<0.05).

FIG. 3 is representative of flow cytometry indicating hESC to epithelial to mesenchymal changes. A) Samples were examined by flow cytometry at p0 (passage 0 or pluripotence) and p1 (passage 1 @˜30 d). B) Protein expression was compared between passages 1 (p1) and 7 (p7). (*p<0.05)

FIG. 4. shows that hES-MC are osteogenic and chondrogenic, but not adipogenic. hES-MC and BM-hMSC were subject to MSC three lineage differentiation protocols. For negative controls, both cell types were cultured in normal growth media. A) Osteogenic conditions and von Kossa staining. B) Chondrogenic conditions and Alcian blue staining. C) Adipogenic conditions and Oil Red-O staining. 10×

FIG. 5 shows hES-MC contract and remodel collagen I lattice. Keloid fibroblasts (KF) and derived hES-MC were seeded into rat tail collagen I lattices floating for 7 d. A) Bright field images show the degree of contraction and remodeling compared to the original lattice size (No Cells, NC). B) Contraction quantification.

FIG. 6 shows that TGF-β1, but not PDGF-B, induce αSMA expression in hES-MC. hES-MC were plated in 10 ng/ml of PDGF-B or TGF-131 for 12 d then immunostained for αSMA (green), F-actin (red) and DAPI (blue). 40×.

### OBJECTS OF THE INVENTION

It is an object of the invention to provide a method of producing human mesenchymal stern cells (hMSCs) or human mesenchymal-like stem cells (hMSC-like).

It is an object of the invention to provide human mesenchymal stem cells (hMSCs) or human mesenchymal-like stem cells (hMSC-like) which have may be further differentiated to produce cells for tissue engineering and for cell based bioassays for disease research and drug screening.

Any one or more of these and/or other objects of the present invention may be readily gleaned from a description of the

### BRIEF DESCRIPTION OF THE INVENTION

The present invention, in broadest terms, is directed to differentiating pluripotent stem cells to epithelial cells (in particular, human embryonic stem cell derived epithelial cells) and the epithelial cells to mesenchymal cells. The mesenchymal cells so produced may be further differentiated into bone cells, cartilage cells and smooth muscle, including vascular tissue and heart tissue.

Thus, in certain aspects, the present invention is directed to a method of producing human mesenchymal stem cells (hMSCs) or human mesenchymal-like stem cells from human embryonic stem cells (hESCs) comprising:

Exposing PSCs (especially hESCs) in culture to a stem cell conditioning medium, optionally on a substrate or differentiation protein to make the cells confluent;

Exposing the confluent PSCs (especially, hESCs) to a differentiation medium optionally on a substrate or differentiation protein wherein said medium comprises effective amounts of fibroblast growth factor, especially basic fibroblast growth factor (bFGF), vascular endothelial growth factor (VEGF) and insulin-like growth factor (IGF), especially IGF-1 (including recombinant versions of IGF-1 such as R³-IGF-1 and optionally, epidermal growth factor (EGF) and/or hydrocortisone for a period of between 1 and 25 days, between about 1 and 20 days, between about 2 and 18 days, about 2 and 17 days, about 3 and 14 days, about 5 and 16 days, about 3 and 15 days, about 6 and 15 days, about 10 and 20 days) to produce a population (preferably, in a uniform sheet) of pluripotent stem cell derived epithelial cells (preferably human embryonic stem cell derived epithelial cells or hESC-EC);

Optionally isolating said stem cell derived epithelial cells (hESC-ECs);

Exposing the stem cell derived epithelial cells, including said optionally isolated stem cell derived epithelial cells (preferably, hESC-EC) to a differentiation medium comprising effective amounts of fibroblast growth factor, especially basic fibroblast growth factor (bFGF), vascular endothelial growth factor (VEGF) and insulin-like growth factor (IGF), especially IGF-1 (including recombinant versions of IGF-1 such as R³-IGF-1 and optionally, epidermal growth factor (EGF) and/or hydrocortisone for a period of at least about 2-5 days (including at least about 2 days, at least about 4 days, about 5 to about 10 days, about 5 to about 15 days, about 7 to about 18 days, about 7 to about 15 days, about 5 to 20 days) effective to differentiate said stem cell derived epithelial cells (preferably, hESC-EC) to stem cell derived mesenchymal cells (preferably hESC-MC); and

Optionally, isolating said mesenchymal cells and/or further differentiating said mesenchymal cells into bone, cartilage and smooth muscle tissue, including vascular tissue and heart tissue by exposing the hESC-MCs to differentiation medium (as otherwise described herein) for a period at least about 24 hours (1 Day) to about 10 days or more. Of course, one or more of the above steps may be removed or eliminated in order to produce the desired cell population.

It is preferred that the pluripotent stems cells (PSCs) are human embryonic stem cells (hESCs) such that the resulting epithelial cells are human pluripotent stem cell derived epithelial cells (PSC-EC) and in particular, human embryonic stem cell derived epithelial cells (hESC-EC) and pluripotent stem cell derived mesenchymal cells (PSC-MC) human embryonic stem cell derived mesenchymal cells (hESC-MC). In preferred aspects of the invention, the cells (stem cells, epithelial cells and mesenchymal cells) are primate cells and in particular, human cells.

In preferred aspects of the invention, the epithelial cells are differentiated on a substrate or differentiation protein to produce mesenchymal cells and said mesenchymal cells are isolated solely by passaging and collecting said cells without a further isolation step. In preferred aspects of the invention, the pluripotent stem cells (e.g., hESCs) and epithelial cells are differentiated on a substrate or differentiation protein and the resulting epithelial and/or mesenchymal cells are isolated by simply passaging and collecting the cells without any further isolation steps.

The present invention is also directed to human pluripotent stem cell derived epithelial cells (hPSC-EC), and in particular, human embryonic stem cell derived epithelial cells(hESC-EC) and human pluripotent stem cell derived mesenchymal cells (hPSC-MC), and in particular, human embryonic stem cell derived mesenchymal cells(hESC-MC) produced by the method according to the present invention and/or as otherwise characterized herein. Each of these cells may be stored using standard cryopreservation techniques.

Methods of producing bone cells, cartilage cells, smooth muscle cells, including vascular cells and heart cells from mesenchymal cells according to the present invention are also described herein.

Thus, the present inventors have advanced the state of the art by developing, in particular aspects of the present invention, an hESC mono-layer differentiation culture system that does not rely on feeder cells, manual selection or sorting to produce 1) uniform epithelial sheets with mesodermal gene expression patterns that 2) upon passaging undergo apparent epithelial-mesenchymal transition (EMT) to produce highly proliferative and uniform mesenchymal progenitor cells (hES-MC) with 3) functional capabilities to differentiate along osteogenic and chondrogenic lineages, contract collagen I lattices and express αSMA when exposed to TGF-β which can be used to produce bone cells, cartilage cells, smooth muscle cells, including vascular cells and heart muscle cells, which may be used in reconstructive surgery, bioengineering and in diagnostic and analytical systems to identify active bioagents. In addition, the present invention may be used to identify potential anticancer agents, by identifying inhibitors of further differentiation of cells of the present invention.

### DETAILED DESCRIPTION OF THE INVENTION

The following terms shall be used to describe the present invention.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Examples included herein. However, before the present compositions and methods are disclosed and described, it is to be understood that this invention is not limited to specific conditions, or specific methods, etc., as such may, of course, vary, and the numerous modifications and variations therein will be apparent to those skilled in the art.

Standard techniques for growing cells, separating cells, analyzing gene expression, determining cell surface biomarkers and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described by Freshney, R. I., Culture of Animal Cells: A Manual of Basic Technique, 5e. 2007, John Wiley & Sons, Inc., New Jersey;

Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The term “primate Pluripotent Stem Cells” or pPSCs, and “human Pluripotent Stem Cells” or hPSCs, of which “human Embryonic Stem Cells” or hESCs are a subset and subsumed under both terms, are derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm and ectoderm), according to a standard art-accepted test, such as the ability to form teratomas in 8-12 week old SOD mice. The term includes both established lines of stem cells of various kinds, and cells obtained from primary tissue that are pluripotent in the manner described.

Included in the definition of pluripotent stem cells (PSCs, including primate pluripotent stem cells or pPSCs and human pluripotent stem cells or hPSCs) are embryonic cells of various types, especially including human embryonic stem cells (hESCs), described by Thomson et al. (Science 282: 1145, 1998); as well as embryonic stem cells from other primates, such as Rhesus stem cells (Thomson et al., Proc. Natl. Acad. Sci. USA 92: 7844, 1995). Other types of pluripotent cells are also included in the term. Human Pluripotent Stem Cells includes stem cells which may be obtained from human umbilical cord or placental blood as well as human placental tissue. Any cells of primate origin that are capable of producing progeny that are derivatives of all three germinal layers are included, regardless of whether they were derived from embryonic tissue, fetal, or other sources. The pPS cells are preferably not derived from a malignant source. It is desirable (but not always necessary) that the cells be karyotypically normal.

pPS cell cultures are described as “undifferentiated” when a substantial proportion of stem cells and their derivatives in the population display morphological characteristics of undifferentiated cells, clearly distinguishing them from differentiated cells of embryo or adult origin. Undifferentiated pPS cells are easily recognized by those skilled in the art, and typically appear in the two dimensions of a microscopic view in colonies of cells with high nuclear/cytoplasmic ratios and prominent nucleoli. It is understood that colonies of undifferentiated cells in the population will often be surrounded by neighboring cells that are differentiated.

Pluripotent stem cells may express one or more of the stage-specific embryonic antigens (SSEA) 3 and 4, and markers detectable using antibodies designated Tra-1-60 and Tra-1-81 (Thomson et al., Science 282:1145, 1998). Differentiation of pluripotent stem cells in vitro results in the loss of SSEA-4, Tra-1-60, and Tra-1-81 expression (if present) and increased expression of SSEA-1. Undifferentiated pluripotent stem cells typically have alkaline phosphatase activity, which can be detected by fixing the cells with 4% paraformaldehyde, and then developing with Vector Red as a substrate, as described by the manufacturer (Vector Laboratories, Burlingame Calif.) Undifferentiated pluripotent stem cells also typically express Oct-4 and TERT, as detected by RT-PCR.

Another desirable phenotype of propagated pluripotent stem cells is a potential to differentiate into cells of all three germinal layers: endoderm, mesoderm, and ectoderm tissues. Pluripotency of pluripotent stem cells can be confirmed, for example, by injecting cells into severe combined immunodeficient (SCID) mice, fixing the teratomas that form using 4% paraformaldehyde, and then examining them histologically for evidence of cell types from the three germ layers. Alternatively, pluripotency may be determined by the creation of embryoid bodies and assessing the embryoid bodies for the presence of markers associated with the three germinal layers.

Propagated pluripotent stem cell lines may be karyotyped using a standard G-banding technique and compared to published karyotypes of the corresponding primate species. It is desirable to obtain cells that have a “normal karyotype,” which means that the cells are euploid, wherein all human chromosomes are present and not noticeably altered.

The types of pluripotent stem cells that may be used include established lines of pluripotent cells derived from tissue formed after gestation, including pre-embryonic tissue (such as, for example, a blastocyst), embryonic tissue, or fetal tissue taken any time during gestation, typically but not necessarily before approximately 10-12 weeks gestation. Non-limiting examples are established lines of human embryonic stem cells or human embryonic germ cells, such as, for example the human embryonic stem cell lines WA01, WA07, and WA09 (WiCell). Also contemplated is use of the compositions of this disclosure during the initial establishment or stabilization of such cells, in which case the source cells would be primary pluripotent cells taken directly from the source tissues. Also suitable are cells taken from a pluripotent stem cell population already cultured in the absence of feeder cells. Also suitable are mutant human embryonic stem cell lines, such as, for example, BG01v (BresaGen, Athens, Ga.), as well as normal human embryonic stem cell lines such as WA01, WA07, WA09 (WiCell) and BG01, BG02 (BresaGen, Athens, Ga.).

Epiblast stem cells (EpiScs) and induced pluripotent stem cells (iPS) fall within the broad definition of pluripotent cells hereunder and in concept, the technology described in the present application could apply to these and other pluripotent cell types (ie, primate pluripotent cells) as set forth above. EpiScs are isolated from early post-implantation stage embryos. They express Oct4 and are pluripotent. See, Tesar et al, Nature, Vol 448, p. 196 12 Jul. 2007. iPS cells are made by dedifferentiating adult somatic cells back to a pluripotent state by retroviral transduction of four genes (c-myc, Klf4, Sox2, Oct4). See, Takahashi and Yamanaka, Cell 126, 663-676, Aug. 25, 2006.

The term “embryonic stem cell” or “ESC” or “hESCs” refers to pluripotent cell, preferably of primates, including humans (hESCs), which are isolated from the blastocyst stage embryo. Human embryonic stem cell refers to a stem cell from a human and are preferably used in aspects of the present invention which relate to human therapy or diagnosis. The following phenotypic markers are expressed by human embryonic stem cells:
SSEA-3, SSEA-4, TRA-1-60, TRA-1-81, CD9, alkaline phosphatase, Oct 4, Nanog, Rex 1, Sox2 and TERT. See Ginis, et al., Dev. Biol, 269(2), 360-380 (2004); Draper, et al., J. Anat., 200(Pt. 3), 249-258, (2002); Carpenter, et al., Cloning Stem Cells, 5(1), 79-88 (2003); Cooper, et al., J. Anat., 200(Pt. 3), 259-265 (2002); Oka, et al., Mol. Biol. Cell, 13(4), 1274-81 (2002); and Carpenter, et al., Dev. Dyn., 229(2), 243-258 (2004). While any primate pluripotent stem cells (pPSCs), including especially human embryonic stem cells can be used in the present methods to produce mesenchymal cells according to the present invention, preferred pPSCs for use in the present invention include human embryonic stem cells, including those from the cell lines BG01 and BG02, as well as numerous other available stem cell lines, resulting in mesenchymal cells termed human embryonic stem cell derived mesenchymal cells or (hESC-MCs).

Human embryonic stem cells may be prepared by methods which are described in the present invention as well as in the art as described for example, by Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 1995).

The term “confluence” refers to the density of cells grown in culture. A culture of cells which is 10% confluent, is used to describe a population of cells which covers approximately 10% of the surface area of the culture dish (flask) in which the cells are grown. Similarly, a culture of cells which is 90% confluent, is used to describe a population of cells which covers approximately 90% of the surface area of the culture dish (flask) in which the cells are grown. In the present invention, cells are generally grown to at least 50%, about 80-90+% confluence, about 90%, about 90+% confluence before passaging and being subjected to a differentiation step. If a cell culture is deemed confluent, the culture completely covers (approximately 100%) of the culture dish.

The term “differentiation” is used to describe a process wherein an unspecialized (“uncommitted”) or less specialized cell acquires the features of a more specialized cell such as, for example, human embryonic stem cell derived epithelial cell (hESC-EC), human embryonic stem cell derived mesenchymal cell (hESC-MC), or where a more specialized intermediate cell, such as a mesenchymal cell (hES-MC) or epithelial cell (hES-EC) becomes an even more specialized cell such as a bone cell, a cartilage cell or a smooth muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. “De-differentiation” refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

The terms “mesenchymal stem cell” “mesenchymal cell” and “human embryonic stem cell derived mesenchymal cell” (hESC-MC) are used interchangeably to refer to a cell or cells produced according to the present invention. hESC-MCs are dynamic multipotent cells which are characterized as being negative for hepatopoietic (CD34, CD45 and CD133) and endothelial (CD31 and CD146) markers, but positive for markers associated with mesenchymal stem cells (MSC), in particular, (CD73, CD90, C D105 and CD166). Mesenchymal stem cells according to the present invention may be used to produce osteogenic (bone) and chondrogenic (cartilage) tissue, but not adipogenic (fat cell) lineages. The derived hESC-MC are able to remodel and contract collagen I lattice constructs to an equivalent degree as keloid fibroblast control cells, They are storage stable (primarily by cryopreservation) and may be passaged for a number of generations and still remain viable. These cells have significant developmental plasticity. They are not hESCs based on marker profiling.

hESC-MCs according to the present invention may be stabilized for storage through cryopreservation of the cells. These cells may be differentiated to bone cells, smooth muscle cells and cartilage cells, among others.

The hESC-MCs according to the present invention have one or more (at least 4, at least 5 at least 6, at least 10, at least 15, preferably all) of the following characteristics:
They can be cultured for at least 10 passages as a stable cell population
Cells appear mesenchymal and have numerous mesenchymal stem cell markers including CD73, CD90, CD105 and CD166
can be produced from a range of hESC lines including BG01, BG02, WA09
hESC-MCs can be frozen and cryogenically preserved by standard methods
hESC-MCs can be recovered after cryogenic storage, recovered and differentiated
hESC-MCs can be passaged with high plating efficiency (greater than 50% plating efficiency-50% of cells passaged successfully seed down and survive)
do not exhibit the heatopoietic markers CD34, CD45 and CD133 on their cell surface
do not express endothelial markers CD31 and CD146
hESC-MCs are E-cadherin negative
hESC-MCs retain a normal karyotype during passaging
hESC-MCs exhibit a mesenchymal phenotype
hEXC-MCs are able to remodel and contract collagen I lattice constructs to an equivalent degree as keloid fibroblast control cells
TGF-β1, but not PDGF-B induces expression of αSMA
have multipotent differentiation capacity (including osteogenic and chondrogenic)
do not have exhibit lipogenic differentiation capacity when exposed to standard lipogenic conditions (high glucose MEM Alpha, supplemented with ITS+1, sodium pyruvate (10 mM), methyl isobutylxanthine (0.5 mM) and dexamethasone (1 μM)
The may be cultured as a monolayer
They require no selection or isolation techniques including but not limited to genetic markers or phenotypic characterization for a MSC phenotype
They pass through a early mesodermal phenotype and epithelial phenotype prior to forming a MSC
They can be genetically manipulated

The term “human embryonic stem cell derived epithelial cell” or hESC-EC is used to describe a cell having characteristics of epithelial cells which is produced from hESC's after several days during differentiation from human embryonic stem cells hESCs to human embryonic stem cell derived mesenchymal cells (hESC-MC). hESCs, exposed to mesenchymal differentiation medium (as described), will produce, after a day or more, usually after at least about several days (generally, between about 1 and 20 days, between about 2 and 15 days, about 2 and 10 days, about 2 and 14 days, about 3 and 6 days, about 3 and 5 days, about 3 and 9 days, about 1 and 9 days,) a uniform sheet of epithelial cells labeled human embryonic stem cell derived epithelial cells (hESC-EC). hESC-ECs tend to begin formation as early as 1-2 days, and form confluent hESC-ECs as a uniform sheet, often after about 15-20 days in culture. After the formation of confluent hESC-ECs, the cells are passaged and then further cultured where they will form confluent hESC-MCs after several days. Generally, confluent hESC-MCs are formed from hESCs after about 25-30 days, having passed through hESC-ECs which begin forming as early as 1-2 days, and forming a confluent uniform sheet of hESC-ECs after about 10-20 days, more frequently about 15-20 days.

Human embryonic stem cell derived epithelial cells or hESC-ECs, according to the present invention have one or more (at least 4, at least 5 at least 6, preferably all) of the following characteristics:
They can be cultured as a stable cell population
Cells appear in an epithelial layer and exhibit mesodermal gene expression patterns
Cells are positive for the following markers: BMP4, RUNX1, GATA4.
Cells can be produced from a range of hESC lines including BG01, BG02, WA09
Cells express E-cadherin (E-cadherin⁺)
Cells express low levels of CD90 (CD90^{low}).
Can be isolated, frozen and cryogenically preserved by standard methods
Can be recovered after cryogenic storage, recovered and differentiated
Can be passaged with high plating efficiency (greater than 50% plating efficiency-50% of cells passaged successfully seed down and survive)
hESC-ECs retain a normal karyotype during passaging
Have multipotent differentiation capacity (epithelial and mesenchymal-like (hESC-MC)
They may be cultured as a monolayer
They require no selection or isolation techniques including but not limited to genetic markers or phenotypic characterization for a EC phenotype
They may be genetically manipulated

As used herein, the terms “differentiation medium”, “cell differentiation medium”, “culture media”, “basal cell medium”, “basal cell media” or “basal media” or “stabilizing medium” are used within the context of its use to describe a cellular growth medium in which (depending upon the additional components used) the hESCs, hESC-MCs, bone, cartilage or smooth muscle tissue are produced, grown/cultured or alternatively, differentiated into more mature cells. Differentiation media are well known in the art and comprise at least a minimum essential medium plus one or more optional components such as growth factors, including fibroblast growth factor (FGF) or basic fibroblast growth factor (bFGF), ascorbic acid, glucose, non-essential amino acids, salts (including trace elements), glutamine, insulin (where indicated and not excluded), transferrin, beta mercaptoethanol, antibiotics (streptomycin, penicillin, etc.) and other agents well known in the art and as otherwise described herein. In the case of differentiation media for human embryonic stem cell derived epithelial cells (hESC-ECs) and mesenchymal cells (hESC-MCs), the basic medium include effective amounts of basic fibroblast growth factor (bFGF), vascular endothelial growth factor (VEGF), and insulin-like growth factor (IGI, especially IGI-1, including a recombinant version of IGI-1, R³-IGI-1) an optionally, epidermal growth factor (EGF) and hydrocortizone. Preferred media includes basal cell media which contains between 1% and 20% (preferably, about 2-10%) fetal calf serum, or for defined medium (preferred) an absence of fetal calf serum and KSR (knockout serum replacement), but including bovine serum albumin (about 1-5%, preferably about 2%). Preferred differentiation medium is defined and is optionally, serum free. In certain embodiments wherein hESC-EC or hESC-MC are produced from human embryonic stem cells (hESCs), the differentiation medium is preferably MCDB 131 with L-glutamine, but without sodium bicarbonate, further supplemented with effective concentrations of basic fibroblast growth factor (bFGF), vascular endothelial growth factor (VEGF), IGF-1 (preferably R³—IGF-1) and optionally, epidermal growth factor (EGF) and/or hydrocortizone.

In the case of differentiation medium for producing bone (osteogenic) cells from hESC-MCs of the present invention, an exemplary differentiation medium is a minimum essential medium (e.g. MEM Alpha) supplemented with fetal bovine serum (about 1-20%, about 5-15%, 10%), dexamethasone (about 10⁻⁸M, about 10⁻⁷ to about 10⁻⁹M), ascorbic acid (about 10-100 μg/ml, about 50 μg/ml) and β-glycerophosphate (10 mM). In producing bone cells according to the present invention, hESC-MCs are grown in the above-described medium, preferably on a support or differentiation protein and preferably feeder-cell free, for a period of at least about 24 hours (1 day) to about 20 days or more.

In the case of differentiation medium for producing cartilage (chondrogenic) cells from hESCs-MCs of the present invention, an exemplary differentiation medium is a minimum essential medium (e.g. MEM Alpha) supplemented with transforming growth factor (TGF, in particular, pTGF-β1- about 1-20 ng/ml, about 5-15 ng/ml, about 10 ng/ml) dexamethasone (preferably about 25-200 nM, about 50-150 nM, about 100 nM), ascorbic acid 2-phosphate (about 10-100 μg/ml, about 25-75 μg/ml, about 50 μg/ml), thyroxine (about 10-100 ng/ml, about, about 50 ng/ml) and ITS+1 (containing insulin from bovine pancreas (about 1.0 mg/ml), human transferrin (substantially iron-free, about 0.55 mg/ml), and sodium selenite (0.5 μg/ml). In producing cartilage cells according to the present invention, hESC-MCs are grown in the above-described medium, preferably on a support or differentiation protein and preferably feeder-cell free, for a period of at least about 24 hours (1 day) to about 20 days or more.

Conditions for differentiation of hESC-MCs to smooth muscle cell may be found in the experimental section which follows. This approach, as well as other approaches known in the art may be used to produce smooth muscle cells, including vascular tissue and cardiovascular tissue (cardiovascular cells).

By way of further example, other suitable media may be made from the following components, such as, for example, Dulbecco's modified Eagle's medium (DMEM), Gibco #11965-092; Knockout Dulbecco's modified Eagle's medium (KO DMEM), Gibco #10829-018; Ham's F12/50% DMEM basal medium; 200 mM L-glutamine, Gibco #15039-027; non-essential amino acid solution, Gibco 11140-050; β-mercaptoethanol, Sigma #M7522; Gibco #13256-029. Preferred embodiments of media used in the present invention are as otherwise described herein.

A particularly preferred differentiation medium for growing/culturing pPSCs (especially, hESCs) to stabilize the cell culture prior to differentiation is DMEM/F12 (50:50) 2 mM L-glutamine, 0.1 mM MEM non-essential amino acids, containing 20% knockout serum replacement (KSR), 50 U/ml Penicillin, 50 μg/ml. streptomycin (from Gibco), about 2-10 ng/ml, about 3-9 ng/ml, about 4 ng/ml bFGF (R & D Systems).

Differentiation media useful in the present invention are commercially available and can be supplemented with commercially available components, available from Invitrogen Corp. (GIBCO), Cell Applications, Inc. and Biological Industries, Beth HaEmek, Israel, among numerous other commercial sources, including Calbiochem. In preferred embodiments the basic differentiation medium further comprises effective amounts of at least three additional growth factors, namely basic fibroblast growth factor (bFGF, about 0.5-7.5 ng/ml, about 1-5 ng/ml, about 2 ng/ml), vascular endothelial growth factor (VEGF, about 0.5-7.5 ng/ml, about 1-5 ng/ml, about 1 ng/ml) and insulin-like growth factor (IGF, in particular, insulin-like growth factor 1 such as a recombinant version of IGF-1, e.g., R³-IGF-1, in general, about 0.5-7.5 ng/ml, about 1-5 ng/ml, about 2 ng/ml) and optionally, epidermal growth factor (EGF, about 5-15 ng/ml, about 8-12 ng/ml, about 10 ng/ml) and/or hydrocortisone (about 0.5-7.5 μg/ml, about 1-5 μg/ml, about 1 μg/ml, are added to the cell media in which a stem cell is cultured and differentiated into a human embryonic stem cell derived epithelial cell (hESC-MC) or mesenchymal cell (hESC-EC). Serum, such as fetal bovine serum (FBS) is also an optional component at a level ranging from about 1% to about 15-20%, about 2% to about 10%, about 3% to about 7.5%, about 5%). It is noted that serum may be avoided in producing cells according to the present invention. One of ordinary skill in the art will be able to readily modify the cell media to produce any one or more of the target cells pursuant to the present invention. By way of reference, cell differentiation medium is essentially synonymous with basal cell medium but is used within the context of a differentiation process and includes cell differentiation agents as otherwise described herein to differentiate cells (KESC into hESC-EC or hESC-MC, hESC-EC or hESC-MC into other cells such as bone cells, cartilage cells, smooth muscle cells, including heart muscle cells.

A differentiation medium for use in the present invention includes MCDB 131 MEDIUM with L-Glutamine and without Sodium Bicarbonate. It is available from Sigma Aldrich and contains the following components:

| Components | g/L |
|---|---|
| Ammonium Metavanadate | 0.000000585 |
| Calcium Chloride anhydrous | 0.1775 |
| Cupric Sulfate•5 H2O | 0.000001249 |
| Ferrous Sulfate•7 H2O | 0.000278 |
| Magnesium Sulfate (anhydrous) | 1.204 |
| Manganese Sulfate | 0.000000151 |
| Molybdic Acid•4 H2O (ammonium) | 0.000003708 |
| Nickel Chloride•6 H2O | 0.000000071 |
| Sodium Phosphate Dibasic | 0.071 |
| Sodium Chloride | 6.4284 |
| Potassium Chloride | 0.2982 |
| Sodium Metasilicate•9 H2O | 0.002842 |
| Sodium Selenite | 0.000005187 |
| Zinc Sulfate•7 H2O | 0.000000288 |
| L-Alanine | 0.00267 |
| L-Arginine•HCl | 0.06321 |
| L-Asparagine•H2O | 0.01501 |
| L-Aspartic Acid | 0.01331 |
| L-Cysteine•HCl•H2O | 0.03512 |
| L-Glutamic Acid | 0.004413 |
| L-Glutamine | 1.461 |
| Glycine | 0.00225 |
| L-Histidine•HCl•H2O | 0.04192 |
| L-Isoleucine | 0.0656 |
| L-Leucine | 0.1312 |
| L-Lysine•HCl | 0.1826 |
| L-Methionine | 0.01492 |
| L-Phenylalanine | 0.03304 |
| L-Proline | 0.01151 |
| L-Serine | 0.03153 |
| L-Threonine | 0.01191 |
| L-Tryptophan | 0.00408 |
| L-Tyrosine•2Na•2 H2O | 0.02252 |
| L-Valine | 0.1171 |
| D-Biotin | 0.000007329 |
| Choline Chloride | 0.01396 |
| Folinic Acid (calcium) | 0.0005115 |
| myo-Inositol | 0.007208 |
| Niacinamide | 0.006105 |
| D-Pantothenic Acid (hemicalcium) | 0.011915 |
| Pyridoxine•HCl | 0.002056 |
| Riboflavin | 0.000003764 |
| Thiamine•HCl | 0.003373 |
| Vitamin B-12 | 0.000013554 |
| Adenine•HCl | 0.0001716 |
| D-Glucose | 1.0 |
| Phenol Red•Na | 0.0124212 |
| Putrescine•2HCl | 0.000000161 |
| Pyruvic Acid•Na | 0.11 |
| DL-6,8-Thioctic Acid | 0.000002063 |
| Thymidine | 0.00002422 |

Another media is EGM2-MV, available from Lonza, Switzerland. The differentiation media which is used to produce epithelial cells and mesenchymal cells from pluripotent stem cells (PSCs), especially including human embryonic stem cells (hESCs) is supplemented with fibroblast growth factor (preferably, basic fibroblast growth factor or bFGF), vascular endothelial growth factor (VEGF), insulin-like growth factor (especially IGF-1, including recombinant versions such as R³-IGF-1) and optionally, epidermal growth factor (EGF) and/or hydrocortisone, all in effective amounts.

Stabilizing medium or conditioning medium is a basal cell medium which is used either before or after a differentiation step in order to grow (to some appropriate level of confluence) and/or stabilize a cell line for further use. It is a cell growth or culture medium, but does not contain growth factors which would otherwise facilitate differentiation of cells. One could also use Mesenchymal Stem Cell growth media available from Invitrogen, Hycon and Millipore and the hESC-MC will proliferate in these media as well. Thus, once the cells are differentiated to hESC-MCs, further proliferation of the cells does not require growth factors, although serum is preferably included. Culture medium is essentially the same as stabilizing medium, but refers to media in which a pluripotent (in particular, hESC) or other cell line is grown or cultured prior to differentiation. In general, as used herein, cell differentiation medium and stabilizing medium may include essentially similar components of a basal cell medium, but are used within different contexts and may include slightly different components in order to effect the intended result of the use of the medium.

Pluripotent stem cells, especially human embryonic stem cells, also may be cultured on a layer of feeder cells (e.g., mitotically inactivated murine embryonic fibroblasts, MEF) that support the pluripotent stem cells in various ways which are described in the art. Alternatively, pluripotent stem cells are cultured in a culture system that is essentially free of feeder cells, but nonetheless supports proliferation of pluripotent stem cells without undergoing substantial differentiation. The growth of pluripotent stem cells in feeder-free culture without differentiation is supported using a medium conditioned by culturing previously with another cell type. Alternatively, the growth of pluripotent stem cells in feeder-free culture without differentiation is supported using a chemically defined medium. These approaches are well known in the art. In preferred aspects of the present invention, the cells are grown in feeder cell free medium.

Approaches for culturing pluripontent stem cells on a layer of feeder cells are well known in the art. For example, Reubinoff et al. (Nature Biotechnology 18: 399-404 (2000)) and Thompson et al. (Science 6 Nov. 1998: Vol. 282. no. 5391, pp. 1145-1147) disclose the culture of pluripotent stem cell lines from human blastocysts using a mouse embryonic fibroblast feeder cell layer. Richards et al, (Stem Cells 21: 546-556, 2003) evaluated a panel of 11 different human adult, fetal and neonatal feeder cell layers for their ability to support human pluripotent stem cell culture and concluded that the human embryonic stem cell lines cultured on adult skin fibroblast feeders retain human embryonic stem cell morphology and remain pluripotent.

US20020072117 discloses cell lines that produce media that support the growth of primate pluripotent stem cells in feeder-free culture. The cell lines employed are mesenchymal and fibroblast-like cell lines obtained from embryonic tissue or differentiated from embryonic stem cells. US20020072117 also discloses the use of the cell lines as a primary feeder cell layer. In another example, Wang et al (Stem Cells 23: 1221-1227, 2005) disclose methods for the long-term growth of human pluripotent stem cells on feeder cell layers derived from human embryonic stem cells. In another example, Stojkovic et al (Stem Cells 2005 23: 306-314, 2005) disclose a feeder cell system derived from the spontaneous differentiation of human embryonic stem cells. In a further example, Miyamoto et al (+22: 433-440, 2004) disclose a source of feeder cells obtained from human placenta. Amit et al (Biol. Reprod 68: 2150-2156, 2003) discloses a feeder cell layer derived from human foreskin. In another example, Inzunza et al (Stem Cells 23: 544-549, 2005) disclose a feeder cell layer from human postnatal foreskin fibroblasts.

Approaches for culturing pPSCs in media, especially feeder-free media, may be used. These are known in the art See, U.S. Pat. No. 6,642,048, U.S. Pat. No. 6,642,048, US20050233446, WO2005065354, WO2005086845 and WO2005014799. US20070010011, discloses a chemically defined culture medium for the maintenance of pluripotent stem cells. Relevant portions of these references are incorporated by reference herein.

An alternative culture system employs serum-free medium supplemented with growth factors capable of promoting the proliferation of embryonic stem cells. For example, Cheon et al (BioReprod DOI: 10.1095/biolreprod. 105.046870, Oct. 19, 2005) disclose a feeder-free, serum-free culture system in which embryonic stem cells are maintained in unconditioned serum replacement (SR) medium supplemented with different growth factors capable of triggering embryonic stem cell self-renewal. In another example, Levenstein et al (Stem Cells 24: 568-574, 2006) disclose methods for the long-term culture of human embryonic stem cells in the absence of fibroblasts or conditioned medium, using media supplemented with bFGF. In still another example, US20050148070 discloses a method of culturing human embryonic stem cells in defined media without serum and without fibroblast feeder cells.

In the present invention, the cells are preferably grown feeder cell free on a cellular support or matrix, as adherent monolayers, rather than as embryoid bodies or in suspension. In the present invention, the use of laminin as a cellular support is preferred (from Sigma, at about 1 μg/cm²). Cellular supports useful in the present invention preferably comprise at least one differentiation protein. The term “differentiation protein” or “substrate protein” is used to describe a protein which is used to grow cells and/or to promote differentiation (also preferably attachment) of an embryonic stem cell, hESC-EC or hESC-MC. Differentiation proteins which are preferably used in the present invention include, for example, an extracellular matrix protein, which is a protein found in the extracellular matrix, such as laminin, tenascin, thrombospondin, and mixtures thereof, which exhibit growth promoting and contain domains with homology to epidermal growth factor (EGF) and exhibit growth promoting and differentiation activity. Other differentiation proteins which may be used in the present invention include for example, collagen, fibronectin, vibronectin, polylysine, polyornithine and mixtures thereof. In addition, gels and other materials such as methylcellulose of other gels which contain effective concentrations of one or more of these embryonic stem cell differentiation proteins may also be used. Exemplary differentiation proteins or materials which include these differentiation proteins include, for example, BD Cell-Tak™ Cell and Tissue Adhesive, BD™ FIBROGEN Human Recombinant Collagen I, BD™ FIBROGEN Human Recombinant Collagen III, BD Matrigel™ Basement Membrane Matrix, BD Matrigel™ Basement Membrane Matrix High Concentration (HC), BD™ PuraMatrix™ Peptide Hydrogel, Collagen I, Collagen I High Concentration (HC), Collagen II (Bovine), Collagen III, Collagen IV, Collagen V, and Collagen VI, among others. The preferred material for use in the present invention includes Matrigel™ and Geltrex™.

A preferred composition/material which contains one or more differentiation or substrate proteins is laminin substrate (from Sigma) at about 1 μg/cm²). Potential other materials useful as cellular support or matrix includes BD Matrigel™ Basement Membrane Matrix. This is a solubilized basement membrane preparation extracted from the Engelbreth-Holm-Swarm (EHS) mouse sarcoma, a tumor rich in ECM proteins. Its major component is laminin, followed by collagen IV, heparan sulfate, proteoglycans, entactin and nidogen.

The pluripotent stem cells (in particular, hESCs) are preferably plated onto the differentiation or substrate protein. The pluripotent stem cells may be plated onto the substrate in a suitable distribution and in the presence of a medium that promotes cell survival, propagation, and retention of the desirable characteristics. All these characteristics benefit from careful attention to the seeding distribution and can readily be determined by one of skill in the art.

As used herein, the term “activate” refers to an increase in expression of a marker which is found in one or more of the cells produced in the present invention, in particular, human embryonic stem cell derived mesenchymal cells (hES-MC). These cells exhibit particular utility in producing bone cells, smooth muscle cells and cartilage cells and can be used in tissue engineering, reconstructive surgery, for repairing bone, for treating heart disease and vascular degeneration and for cell based assays for identifying potential drugs to be used to potentiate or inhibit the differentiation process (anticancer agents), treating heart disease, kidney degeneration, the repair of bone and vascular degeneration.

As used herein when referring to a cell, cell line, cell culture or population of cells, the term “isolated” refers to being substantially separated from the natural source of the cells such that the cell, cell line, cell culture, or population of cells are capable of being cultured in vitro. In addition, the term “isolating” may be used to refer to the physical selection of one or more cells out of a group of two or more cells, wherein the cells are selected based on cell morphology and/or the expression of various markers. It is noted herein that in preferred aspects of the present invention, one of the principal benefits is that isolation of cells, because of the levels of confluence and population consistency, do not require a separate isolation technique or step. Within this context, the term “isolating” may simply refer to the passaging of cells without further isolation steps being used to provide unexpected consistency of the final isolated cell population.

As used herein, the term “express” refers to the transcription of a polynucleotide or translation of a polypeptide (including a marker) in a cell, such that levels of the molecule are measurably higher in or on (cell surface) a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, the term “Markers” describe nucleic acid or polypeptide molecules that are differentially expressed in a cell of interest, in particular, human embryonic stem cell derived mesenchymal cells (hES-MC) and human embryonic stem cell derived epithelial cells (hES-EC). In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

As used herein, the term “contacting” (i.e., contacting a cell with a compound) is intended to include incubating the compound and the cell together in vitro (e.g., adding the compound to cells in culture). The term “contacting” is not intended to include the in vivo exposure of cells to a differentiation agent that may occur naturally in a subject (i.e., exposure that may occur as a result of a natural physiological process). The step of contacting the cell with differentiation medium and one or more factors as described herein can be conducted in any suitable manner. For example, the cells may be treated in adherent culture as an adherent layer, as embryoid bodies or in suspension culture, although the use of adherent layers are preferred because they provide an efficient differentiation process oftentimes providing differentiation to a target cell population (human embryonic stem cell derived mesenchymal cell or hES-MC) of 90% or more. It is understood that the cells contacted with the differentiation agent may be further treated with other cell differentiation environments to stabilize the cells, or to differentiate the cells further, for example to produce smooth muscle cells, bone cells and cartilage cells, among others.

In the case of producing human embryonic stem cell derived mesenchymal cells (hES-MC) from human embryonic stem cells, human embryonic stem cells are differentiated in a medium as otherwise disclosed herein comprising a cell differentiation medium and the following growth factors: basic fibroblast growth factor (bFGF), vascular endothelial growth factor (VEGF), epidermal growth factor (EGF), insulin-like growth factor (IGF-1), preferably the R³ analog of IGF-1 (R³-IGF-1- the long R³ analog of IGF-1).

As used herein, the term “differentiation agent” refers to any compound or molecule that induces a cell such as a pluripotent stem cell (PSC), especially hESC's, pluripotent, especially embryonic stem cell derived epithelial cells, and in particular human embryonic stem cell derived epithelial cells (hES-EC), pluripotent, especially embryonic stem cell derived mesenchymal cells, and in particular human embryonic stem cell derived mesenchymal cells (hES-MC) to partially or terminally differentiate. While the differentiation agent may be as generally described below and may reflect the agent in producing an intermediate and final/or differentiation cell, the term is not limited thereto. The term “differentiation agent” as used herein includes within its scope a natural or synthetic molecule or molecules which exhibit(s) similar biological activity.

The term “effective” is used to describe an amount of a component, compound or composition which is used or is included in context in an amount and for a period sufficient to produce an intended effect. By way of example, an effective amount of a differentiation agent is that amount which, in combination with other components, in a differentiation medium will produce the differentiated cells desired. In all instances, except as otherwise stated herein, components are used in effective amounts within the context of their use in the present invention.

The term “passaged” or “passaging” is used to describe the process of splitting cells and transferring them to a new cell vial for further growth/regrowth or for storage. The preferred adherent cells (or even embryoid bodies) according to the present invention may be passaged using enzymatic (trypsinase, Accutase™, collagenase) passage, manual passage (mechanical, with, example, a spatula or other soft mechanical utensil or device) and other non-enzymatic methods, such as cell dispersal buffer. It is noted that after passaging, cells are then further grown and/or differentiated in cell culture flasks by coating approximately 1×10³ cells/cm² to about 5×10⁷ cells/cm² per flask, about 5×10³ cells/cm² to about 1×10⁷ cells/cm² per flask, about 1×10⁴ cells/cm² to about 5×10⁶ cells/cm² per flask, about 2.5×10⁴ cells/cm² to about 1×10⁶ cells/cm² per flask, about 4×10⁴ cells/cm² to about 5×10⁵ cells per flask (preferably, a T75 flask). Cells are generally grown to at least about 50% confluence, preferably about 75-90% confluence, preferably about 90% confluence. In most instances after reaching confluence, the cells are then isolated or passaged and further grown and/or differentiated. Unless otherwise specifically stated, in most instances, cells are passaged after 2-4 (2-3) days of being grown or cultured in medium.

The present invention contemplates a composition comprising a population of isolated differentiated mammalian cells, in particular, pluripotent stem cell derived epithelial cells, in particular human pluripotent stem cell derived epithelial cells (hPSC-EC), in particular, human embryonic stem cell derived epithelial cells (hESC-EC) or pluripotent stem cell derived mesenchymal cells, in particular human pluripotent stem cell derived mesenchymal cells (hPSC-MC), in particular, human embryonic stem cell derived epithelial cells (hESC-MC). In certain embodiments of the invention, greater than approximately 35%, 40%, 45%, 50%, 55%, 60%, 65%, 67%, 70%, 72%, 74%, 75%, 76%, 77%, 78%, 79%, 80%, 90% or greater than 90% of the cells are epithelial cells or mesenchymal cells. Preferably, composition comprises a population of cells at least about 50% epithelial cells, up to 70-80%, 90% or more. The cell population (epithelial cells or mesenchymal cells) is storage stable and may be cryopreserved to that end. Cryopresevation techniques well known in the art may be used.

Human embryonic stem cell derived mesenchymal cells may be further differentiated to bone, cartilage and smooth muscle cells, including vascular cells or heart muscle cells using approaches well known in the art. See, Rojas, et al., Development 2005; 132:3405-3417 and Ullmann, et al., Cancer Res 2007; 67:11254-11262. Generally, aliquots of 200,000 cells or more of cells may be distributed in 15-ml conical tubes and centrifuged 5 min at 600×g. Sedimented cells are cultured in the tubes (or in cell culture flasks) with loosened caps to allow gas exchange. Cells form a spherical mass on the bottom of the tube by 24 h or layers of cells by 24 h or more (flasks) of culture.

In the case of differentiation medium for producing bone (osteogenic) cells from hESC-MCs of the present invention, an exemplary differentiation medium is a minimum essential medium (e.g. MEM Alpha) supplemented with fetal bovine serum (about 1-20%, about 5-15%, 10%), dexamethasone (about 10⁻⁸M, about 10⁻⁷ to about 10⁻⁹M), ascorbic acid (about 10-100 μg/ml, about 50 μg/ml) and β-glycerophosphate (10 mM). In producing bone cells according to the present invention, hESC-MCs are grown in the above-described medium, preferably on a support or differentiation protein and preferably feeder-cell free, for a period of at least about 24 hours (1 day) to about 20 days or more. Bone cell differentiation is evidenced by accumulation of phosphates and carbonates, as demonstrated using the von Kossa silver reduction method [4]. Cultures or cryosections were fixed with 4% formaldehyde, exposed to 5% silver nitrate solution and immediately exposed to direct UV light 197 for 45-60 minutes. Specimens were then washed and incubated for 2-3 min in 5% sodium thiosulfate solution. Expression of alkaline phosphatase (AP) was assessed by a commercial kit (Vector Red Alkaline Phosphatase Substrate Kit I, Vector Laboratories, Burlingame, Calif.).

In the case of differentiation medium for producing cartilage (chondrogenic) cells from hESCs-MCs of the present invention, an exemplary differentiation medium is a minimum essential medium (e.g. MEM Alpha) supplemented with transforming growth factor (TGF, in particular, pTGF-β1- about 1-20 ng/ml, about 5-15 ng/ml, about 10 ng/ml) dexamethasone (preferably about 25-200 nM, about 50-150 nM, about 100 nM), ascorbic acid 2-phosphate (about 10-100 μg/ml, about 25-75 μg/ml, about 50 μg/ml), thyroxine (about 10-100 ng/ml, about, about 50 ng/ml) and ITS+1 (containing insulin from bovine pancreas (about 1.0 mg/ml), human transferrin (substantially iron-free, about 0.55 mg/ml), and sodium selenite (0.5 μg/ml). In producing cartilage cells according to the present invention, hESC-MCs are grown in the above-described medium, preferably on a support or differentiation protein and preferably feeder-cell free, for a period of at least about 24 hours (1 day) to about 20 days or more. Differentiation of mesenchymal cells to cartilage cells was evidenced by acidic mucopolysaccharides present in cartilage tissue as stained with alcian blue 8GX (Sigma Chemical, St. Louis, Mo., USA). Briefly, cryosections are 206 fixed with 3% acetic acid and stained with alcian blue solution (1% w/v alcian blue in 3% acetic acid, pH 2.5) for 30 min. After washing, slides are mounted with 90% glycerol and inspected with a transmitted light microscope. Photographs are taken with a digital camera (Qimaging Ratiga 1300, Qimaging, Burnaby, BC, Canada) mounted on the microscope.

The following abbreviations, among others, are also used to describe the present invention: EMT—epithelial-mesenchymal transition, MSC—mesenchymal stem cell, αSMA—a smooth muscle actin, SMC—smooth muscle cell, EC—epithelial cell

The invention shall be further described in the examples which are presented hereinbelow. These examples are for edification and are not to be construed as limiting the scope of the invention in any way.

### Materials and Methods

### Cell Culture

Karyotypically normal human embryonic stem cell lines BG01 (Bresagen) and WA09 (WiCell) were cultured in 20% KSR media (DMEM/F12, 2 mM L-glutamine, 0.1 mM MEM non-essential amino acids, 50 U/ml penicillin, 50 μg/ml streptomycin, 20% knock-out serum replacement (KSR)) (all from Gibco) and 4 ng/ml basic fibroblast growth factor (bFGF, R & D Systems). Cells were cultured on Mitomycin-C (Sigma) mitotically inactivated murine embryonic fibroblasts (MEF), manually dissociated and passaged to new feeder layers every 4-5 days [23]. For feeder-free culture of hESC, cells grown on MEFs were washed once with PBS⁻⁻ (without Ca²⁺ and Mg²⁺) then incubated with 0.25% trypsin (Gibco) until the MEF layer began to lift off the dish. The floating MEF layer was discarded after agitating it to release adherent stem cells which were collected, centrifuged and resuspended in MEF conditioned media (CM). CM was prepared by placing 20% KSR media on MEF for 24 h then supplementing the collected media with an additional 4 ng/ml of bFGF [24]. Cells were plated on tissue culture dishes coated with laminin substrate (Sigma, 1 μg/cm²) and grown to ˜90% confluence. The cells were passaged at least 3 times to minimize MEF contamination. Keloid fibroblasts were purchased from ATCC grown in DMEM, penicillin/streptomycin (Gibco) and 10% FBS (Hyclone). Bone marrow derived MSC (BM-hMSC) were purchased from Lonza and grown in proprietary MSC media (Lonza).

### Differentiation Procedure and Culture

When hESC cultured without feeders as described above reached ˜90% confluence the 100 mm dishes were washed with PBS⁺⁺ (with Ca²⁺ and Mg²⁺) and replaced with 10 ml of fresh EGM2-MV (Lonza; 5% FBS, proprietary EBM2 basal media and concentrations of bFGF, VEGF, EGF and R³-IGF-1 [25]). The media was changed every 2-3 days over a period of 20-30 days. After transition from hESC to epithelial sheet was completed the cells were trypsin passaged to a T75 flask and grown to confluence. To expand the initial cell culture, cells were passaged and seeded at a target density of approximately 4×10⁴ cells/cm² per flasks. For subsequent culture for experimentation, cells were subcultured at 10⁶ cells/T75 flask (˜1.3×10⁵ cells/cm²) and grown to confluence over 5-7 d.

### Light Microscopy

Phase contrast and bright field images were acquired using a Nikon Eclipse TE 2000-S inverted microscope (Nikon) and Image Pro Plus v5.1 (Media Cybernetics). Dark field images were acquired with a Nikon TS100 microscope with attached Nikon Coolpix 4500 digital camera. All image settings were controlled for uniform acquisition between samples.

### Gene Transcription and Quantitative Real-Time PCR

hESC were grown as described over a 30 day period in 6-well plates. Samples were collected for RNA analysis on day 0 (Control) and every 5 days thereafter. Total RNA was isolated using a Qiagen RNeasy kit according to the manufacturer's instructions and quantified using RNA 600 Nano Assay and the Agilent 2100 Bioanalyzer (Agilent Technologies). cDNA was reverse transcribed using Superscript II (Invitrogen). qRT-PCR was processed using an ABI 7900HT in a low-density array previously described [26]. Relative quantification of the gene expression output was performed using Sequence Detection System software (SDS v2.2.1, ABI). The SDS utilizes relative quantification of gene expression by way of the comparative C_{T} method where the relative quantity (RQ)=2⁻ΔΔC_{T}, ΔΔC_{T=}(C_{T.Target} 2 C_{T.Actin})_{Time x} 2 (C_{T.Target} 2 C_{T.Actin})_{Time 0} and C_{T} is defined as the threshold cycle where the target gene surpasses a defined amplification [27]. All genes were normalized to 18 S as a loading control and day 0 as the base line expression. Initial data plots and analysis indicated substantial skewness and non-constant variance of the error terms. As such, a Box-Cox transformation (λ=0), was applied to the RQ values. The ln(RQ) were analyzed using ANOVA (SAS) to determine the significance of the changes in gene expression over the time course. When ANOVA significance was determined (p<0.05), a least square mean (LSM) analysis was performed to examine the effect of day (i.e. each time point) of gene expression (SAS).

### Flow Cytometry

Cells were fixed in 57% ethanol in PBS⁺⁺ for 10 minutes at room temperature. Cells were washed 2× in PBS⁺⁺ and incubated with PBS⁺⁺ with 5% FBS. Antibodies were directed against Oct4 (Santa Cruz), Tra-1-60 (Chemicon), E-cadherin, CD31, CD34, CD45, CD73, CD90, CD146, CD166 (BD Biosciences), CD133 (Miltenyi Biotec) and CD105 (eBioscience). When primary antibodies were not directly conjugated with the fluorophor (PE or FITC), indirect detection was achieved using fluorescently conjugated secondary antibodies Alexa Flour 488 (Molecular Probes). Isotype controls were run to determine non-specific binding. Cells were sorted and analyzed using a FACSCaliber (BD) and FlowJo Cytometry analysis software (Tree Star).

### Lineage Differentiation Assays

Derived hES-MC were tested for three lineage differentiation using modifications of previously published protocols [16,28,29]. Briefly, derived cells were passaged onto 6-well tissue culture plates at a concentration of 2.5×10⁵ cells/well (35 mm) for osteogenic and adipogenic induction. For chondrogenesis, a 10 μl cell suspension micromass (2×10⁷ cells/ml) was allowed to adhere in a 35 mm tissue culture dish for 1 h, then media was added to prevent desiccation. After an overnight incubation at 37° C., 1 ml of proliferation or differentiation media was added to the well. Osteogenic derivation media: DMEM (Low Glucose), 100 nM dexamethasone, 50 μM ascorbic acid, 10 mM β-glycerophosphate (Sigma), 10% FBS (Hyclone) and Pen/Strep (Gibco). Adipogenic media: Derivation: DMEM (High Glucose), Pen/Strep (Gibco), 1 μM dexamethasone, 10 μg/ml insulin, 200 μM indomethacin, 500 μM 3-isobutyl-1-methyl-xanthine (IBMX) (Sigma), 10% FBS (Hyclone); Maintenance: DMEM (HG), Pen/Strep, 10 μg/ml insulin and 10% FBS. Chondrogenic derivation media: DMEM (HG), 100 nM dexamethasone, Pen/Strep, 50 μg/ml ascorbic acid, 40 μg/ml L-proline, ITS+1 supplement, 1 mM sodium pyruvate (Sigma), 10 ng/ml TGFβ-3 (R&D Systems).

### Collagen I Lattice Contraction Assay

Rat tail collagen I (BD Bioscience) was prepared as recommended by the manufacturer to a concentration of 1 mg/ml and cell density of 1.25×10⁵ cells/ml [30]. A 250 μl volume was spotted on to plastic Petri dishes (BD Falcon) and allowed to polymerize for 1 h at 37° C. Afterwards, media was added to the dish and the spot was gently released from the plate with a cell scraper (Sarstadt). The collagen I constructs were cultured for 7 d with the media changed every other day. Images of the construct were acquired using a Nikon TE-1500 dissection microscope with DS-5M (Nikon) camera. Contraction was calculated by averaging the construct length in two perpendicular directions, then taking the average cross-sectional length of the floating construct and normalizing this to the average length of lattice without cells.

### PDGF-B and TGF-β1 Induction of αSMA

hES-MC (B4, E21b, E22h and E28h) were plated at a concentration of 10⁴ cells/cm² onto glass chamber slides (BD Falcon) coated with rat tail collagen I (BD Biosciences) at 5 μg/cm² per the manufacturers' directions. Cells were exposed to low glucose DMEM (Sigma), 10% FBS (Hyclone) with PDGF-B or TGF-β (10 ng/ml each, R&D Systems) or as a negative control EGM2-MV for 12 d. Cells were fixed with 2% PFA (Sigma), permeablized with 0.5% Triton X100 (Fisher Scientific) for 10 min, washed 10 mM glycine for 15 min, blocked for 1 h in PBS⁺⁺ with 3% donkey serum (Jackson ImmunoResearch) and 1% BSA (Sigma). The wells were incubated for 1 h at RT with a monoclonal antibody to αSMA (clone 1A4, Abcam) at 1:500, washed 2 times in block and once in PBS⁺⁺ for 15 min each, incubated for 1 h at RT with donkey anti-mouse AlexaFluor 488 secondary antibody (1:2000, Molecular Probes). The wells were washed 3 times with PBS⁺⁺ then incubated for 30 minutes at RT with Phalloidin conjugated AlexaFluor 546, washed 3 times in PBS⁺⁺ and treated with Prolong Gold with DAPI (Invitrogen). Wells were imaged using an inverted fluorescence microscope with Disc-spinning unit (IX81, Olympus) with a 40× oil objective.

### Results

Monolayer Culture and hESC Formation of Epithelial Phenotype

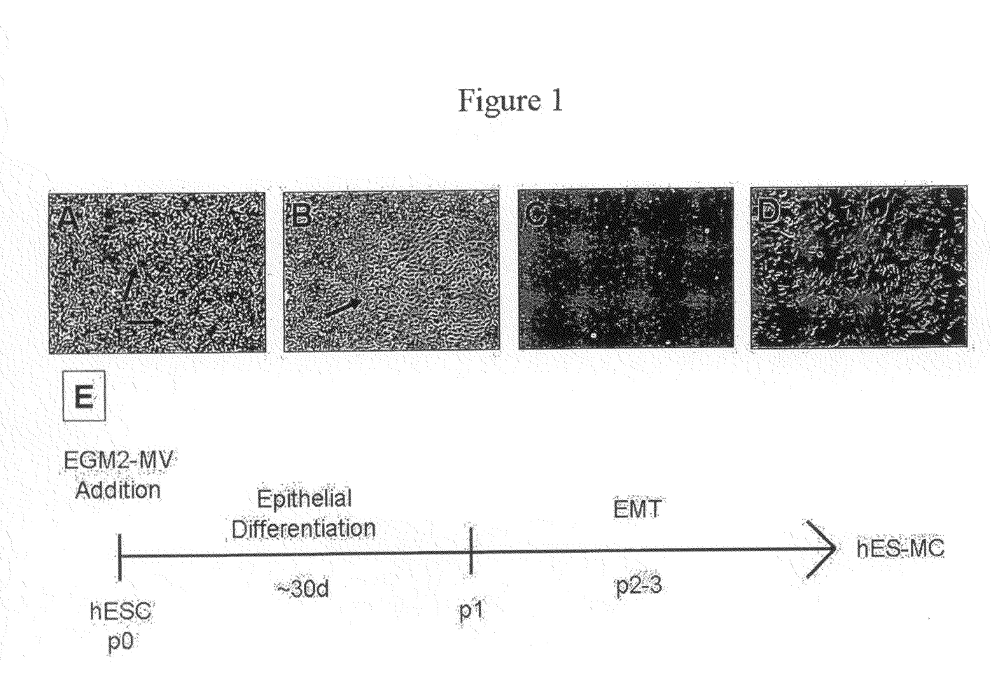

A monolayer culture system was selected because of its advantages over embryoid body differentiation to potentially avoid multiple cell types from multiple germ lineages. Kaufman, et al [31], produced endothelial-like cells from Rhesus monkey ESC in a 2D culture approach by changing from growth media to the proprietary endothelial microvascular media EGM2-MV. Instead of culturing the hESC on MEFs as in Kaufman, et al, we passaged hESC (BG01 and WA09) onto 100 mm tissue culture dishes coated with laminin at 1 μg/cm². The hESC were grown in MEF conditioned media as described until reaching approximately 90% confluence. At that time, the media was changed to EGM2-MV with fresh media being added every 2-3 days over a 20 to 30 day period (FIG. 1). Within five days of changing to EGM2-MV, foci of epithelial cells began to appear (FIG. 1A, Arrow). These foci grew as circular expanding epithelial sheets at multiple points within the dish (FIG. 1B, Arrow) that enlarged until the epithelial phenotype filled the dish (FIG. 1C). A time line for the differentiation procedure is presented in FIG. 1E.

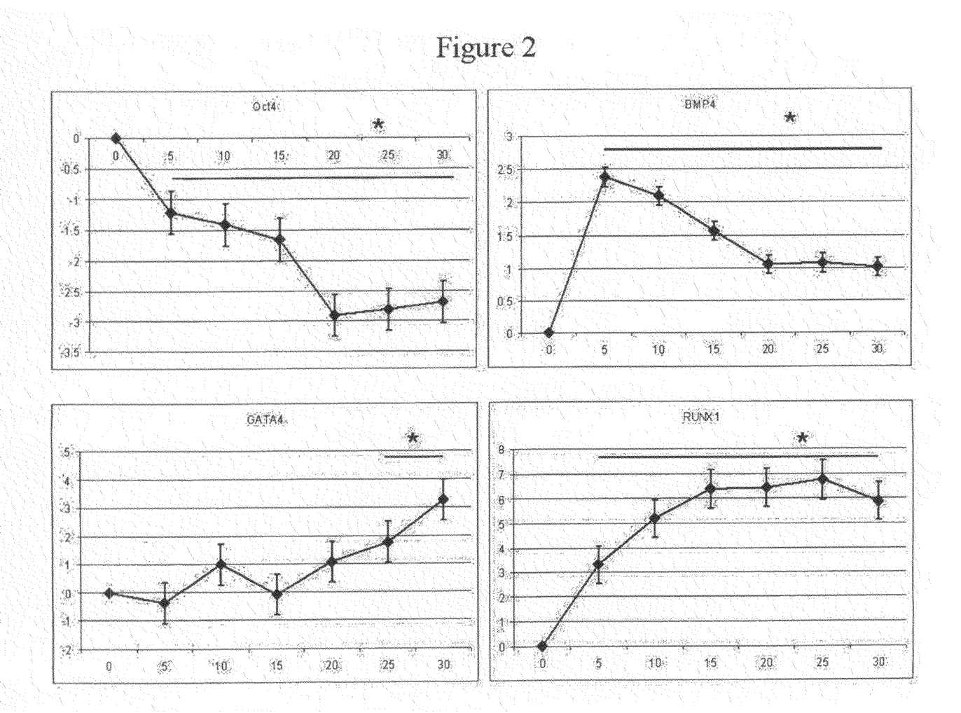

To examine the gene expression of the changing hESC during this process a time course was performed over 30 d where samples were acquired on day 0 (hESC control) and every 5 days thereafter. Samples were processed for RNA isolation and total cDNA reverse transcribed. As expected in any differentiation protocol of hESC, the majority of genes with statistically significant expression changes were pluripotent makers (i.e. POU5F1\Oct4 [FIG. 2], DNMT3B, FGF2, FGFR4, SALL2, SOX2 [data not shown]; p<0.05). Of the 9 genes representing the ectoderm and endoderm, only one, FST (Follistatin, an activin and BMP inhibitor) was significantly different from hESC (p=0.001, data not shown). Three genes in the mesoderm category, BMP4, GATA4 and RUNX1, were significantly up-regulated from hESC (FIG. 2). BMP4 gene expression was maximally up-regulated at day 5 then decreased to a steady-state at days 20 to 30. Both RUNX1 and GATA4 are down-stream targets of BMP4 signaling [32,33] and were up-regulated later than BMP4 indicating transcriptional control by this pathway. This data suggests the hESC may be preferentially differentiating along the mesodermal lineage.

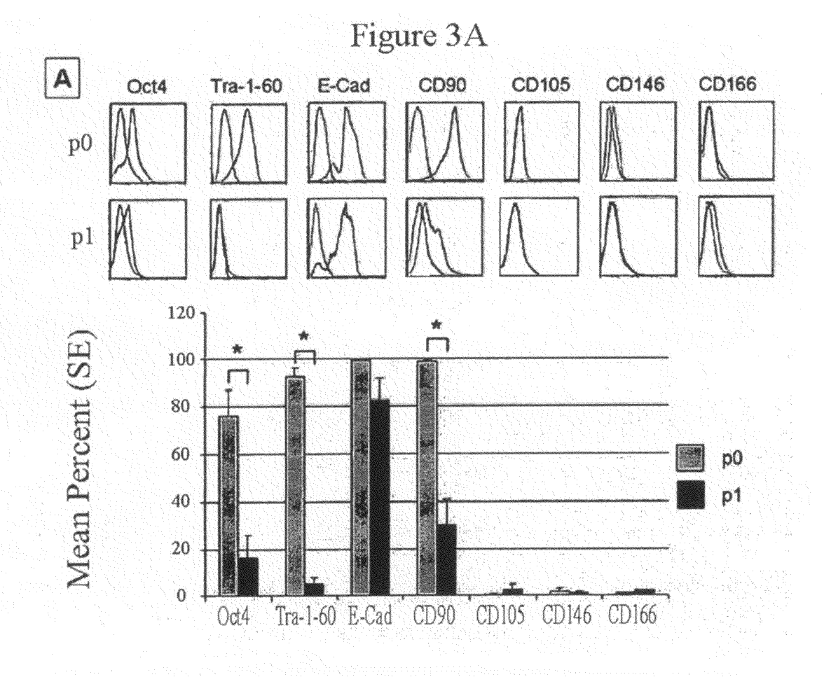

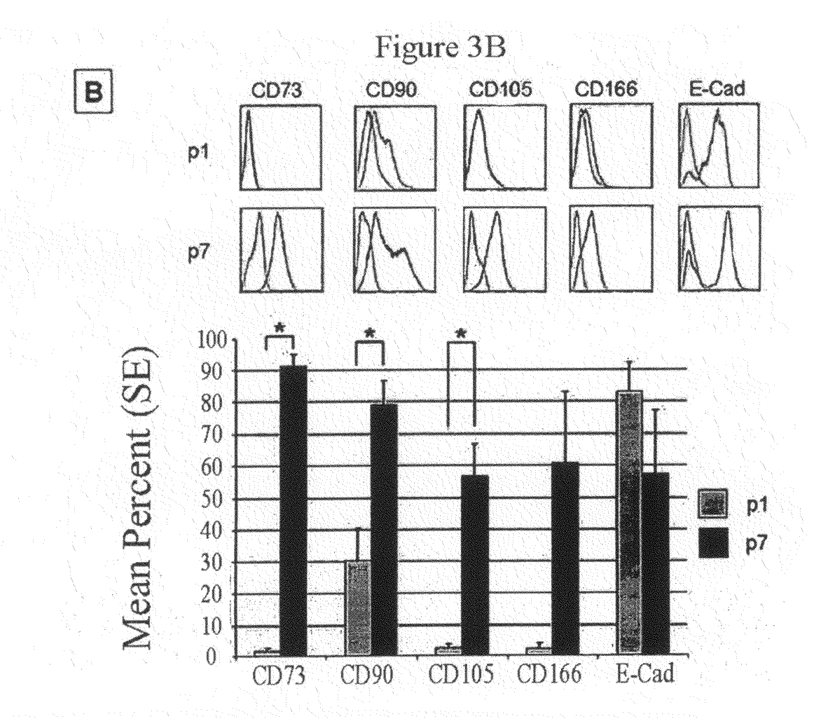

In addition to gene transcription we also examined multiple markers of pluripotence, epithelial and mesenchymal cells by flow cytometry. Samples were collected at the outset of each experiment (p0, hESC control) and at the first passage (p1, ˜30 d) and immunostained for Oct4, Tra-1-60, E-cadherin, CD90 (Thy-1), CD105, CD146 and CD166 (FIG. 3A). At p0 the hESC highly expressed markers shown to be associated with the pluripotent state (Oct4, Tra-1-60, E-Cad and CD90) [34-36]. When the culture was fully differentiated toward the epithelial phenotype (p1), flow cytometry showed significant down-regulation of Oct4 and Tra-1-60, markers more specifically associated with pluripotence. E-cadherin was also found to be expressed at p1 since it is associated with epithelial cells as well as stem cells. On the other hand, besides stem cells, CD90 is expressed in mesenchymal cells such as mesenchymal stem cells (MSC) and fibroblasts [17]. Thus we found down-regulation of CD90 in the epithelial cells. The commonly used endothelial and mesenchymal markers CD105, CD146 and CD166 were not detected in the stem cells (p0) or in the derived epithelial cells (p1). This suggests the 2D monolayer culture of hESC grown on laminin in EGM2-MV differentiate from stem cells through a mesoderm-like state toward an epithelial phenotype.

Derived Epithelial Cells Undergo EMT with Passaging

During differentiation to epithelium the culture is not passaged for ˜20 to 30 d (FIG. 1E). Once the epithelial transition is completed (termed p1) we began to serially passage the cells. After 2 to 3 passages (˜14-21 d) the epithelial phenotype (FIG. 1C) undergoes a transition to a mesenchymal phenotype (FIG. 1D). To examine the expression of mesenchymal markers we again performed flow cytometry and compared expression between the first and seventh passage (p1 vs. p7) (FIG. 3B). At p1 there was minimal expression of the mesenchymal markers CD73, CD105 and CD166 while by p7 all were highly expressed. Both CD73 and CD105 were significantly different (*p<0.05) and while CD166 was not (p=0.08), the trend indicated higher expression in the mesenchymal cells. CD90, which was highly expressed in the undifferentiated stem cells, seemed to undergo a phenotype dependent down-regulation in the epithelial cells and was again up-regulated as the epithelium transitioned to mesenchymal cells (*p<0.05). The stem cell/epithelial marker E-cadherin was found to decrease with transformation, but its change was not significant. This data suggests the epithelial sheet undergoes an EMT-like process with passaging and differentiates into hESC derived mesenchymal cells (hES-MC).

hES-MC are Osteogenic and Chondrogenic, but not Adipogenic

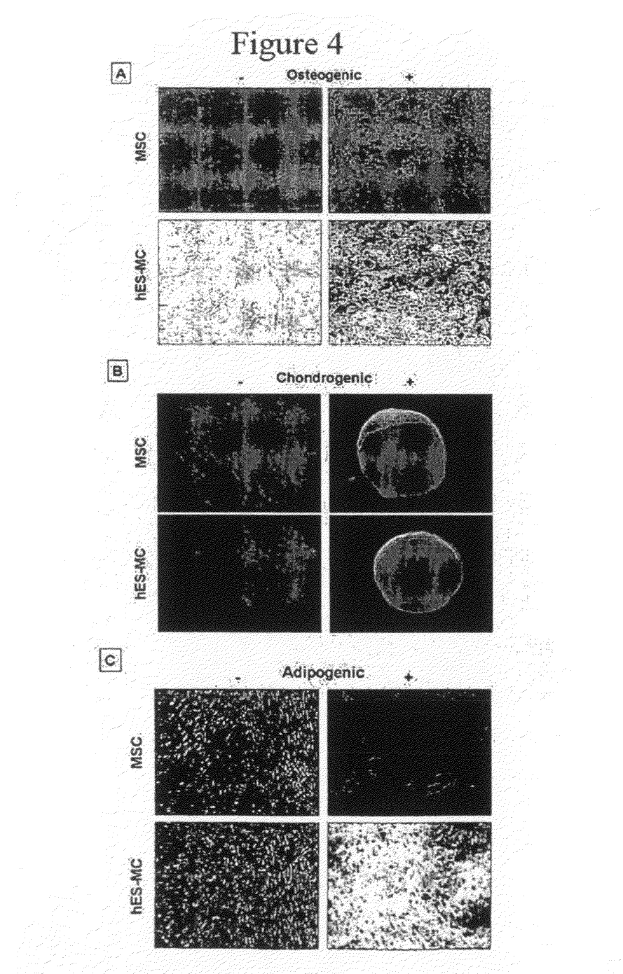

The markers expressed by hES-MC are also expressed by human MSC; therefore the derived cells were tested to see if they possessed tri-lineage capabilities. Standard differentiation techniques were used to determine their ability to transform into osteogenic, chondrogenic and adipogenic cells. As a positive control commercially available human MSC was used and subjected to the differentiation protocols in parallel with the derived cells. When subject to von Kossa staining for calcium detection, both the MSC and the hES-MC cultured in growth media did not form calcium deposits (FIG. 4A, First Column). Under osteogenic conditions both cell lines showed the typical pattern of von Kossa positive staining indicating osteogenic activity (FIG. 4A, Second Column). In the chondrogenic assay, the negative control micromass in normal growth media (FIG. 4B, First Column) spread out on the culture dish loosing its original dome shape and showed no staining of acidic mucopolysaccharides by Alcian blue [37]. When MSC and hES-MC were exposed to chondrogenic media the micromass partially lifted off the plate and formed spherical masses (FIG. 4B, Second Column). Alcian blue showed distinct mucopolysaccharide staining within the cell mass. Though the hES-MC were responsive to differentiation toward osteogenic and chondrogenic lineages, this was not the case for adipogenesis. The growth media cultured cells did not, as expected, form lipid vesicles (FIG. 4C, First Column) for either MSC or hES-MC. When MSC were exposed to adipogenic media they rapidly formed pockets of lipid vesicles that stained positive with Oil Red-0 (FIG. 4C, Second Column, Top). In contrast, the hES-MC showed no vesicle formation or positive Oil Red-0 staining when cultured under the same differentiation conditions (FIG. 4C, Second Column, Bottom). This data shows the derived hES-MC posses some of the differentiation properties of MSC, though not all, suggesting they may be a mesenchymal progenitor cell.

heS-MC Contract Collagen I Lattice

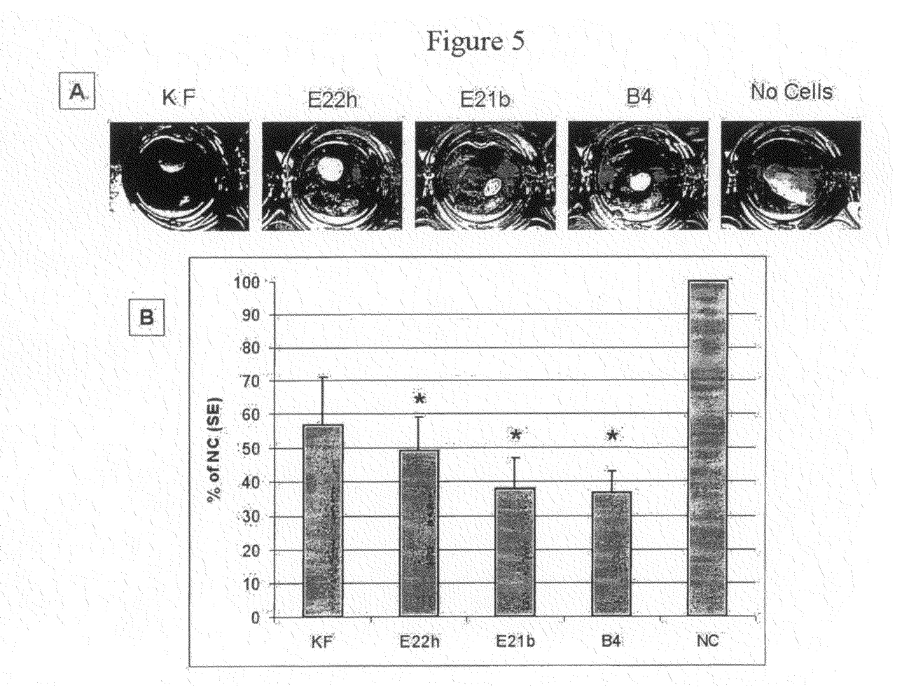

MSC, like fibroblasts, have been shown capable of contracting floating collagen I gels [38,39]. After 7 d post seeding, the floating collagen I constructs were transferred to a 24-well plate, imaged (FIG. 5A) and their lengths measured and normalized to the no-cell (NC) negative control (FIG. 5B). The positive control keloid fibroblasts (KF) were able to remodel and contract the collagen I lattice to approximately 57±14% of the size of the negative control while the hES-MC cell lines were all able to contract the collagen lattice to a greater degree (*p<0.05; E22h=49±9%, E21b=38±9%, B4=37±6%). This suggests the derived hES-MC posses functional abilities to sense and remodel their environment.

TGF-β1, but not PDGF-B, Induces αSMA Expression in hES-MC

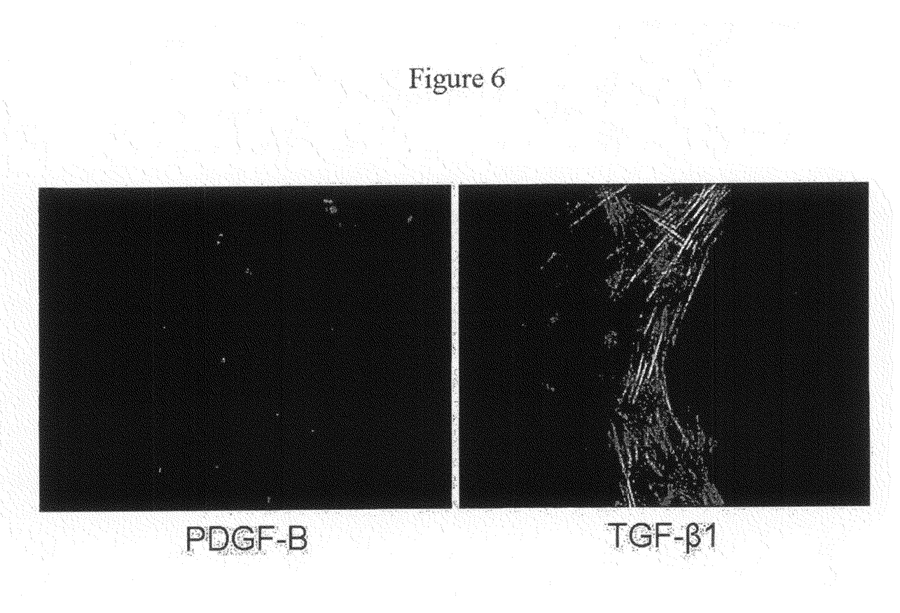

It has been shown that bone marrow derived MSC can be induced by TGF-(β1 to express the early smooth muscle marker αSMA, while PDGF-B does not [40]. Therefore, we plated hES-MC (B4, E21b, E22h and E28h) on collagen I coated chamber slides and exposed them to 10 ng/ml each of TGF-β1 or PDGF-B in low-glucose DMEM with 10% FBS or EGM2-MV as a negative control for 12 days. As can be seen in the representative images in FIG. 6, PDGF-B did not induce the hES-MC to begin expression of αSMA. In contrast, exposure to TGF-131 does cause induction of αSMA expression in some of the cells (αSMA-green, F-Actin-red, DAPI-blue). This evidences the derived hES-MC are responsive to TGF-β1 and may be able to differentiate along the smooth muscle lineage.

### Discussion

The main and novel findings of this study are 1) hESC can form a morphologically uniform epithelium (E-cadherin⁺ CD90^{low}) in 2D culture that can undergo apparent EMT with passaging, 2) the derived cells show gene expression patterns indicating a mesodermal lineage and 3) they posses multiple characteristics of MSC being osteogenic, chondrogenic, but not adipogenic, able to remodel 3D collagen lattice and induced to express αSMA upon exposure to TGF-β1.

The goal was to design a 2D (monolayer) differentiation protocol for epithelial and mesodermal lineages such as endothelial cells from hESC. This was initiated by using a laminin matrix substrate previously shown in the present inventors' lab to facilitate uniform ectodermal differentiation [41,42]. In addition, Kaufman, et al, [31] used EGM2-MV to derive the mesoderm derivative endothelial cells from rhesus monkey ESC. Under these conditions a relatively uniform epithelial sheet of cells was achieved, however they did not express common EC markers (i.e. CD31, vWF, VE-cadherin) [43] or CD146 [31]. Although these cells did not seem to be EC, gene expression data suggested the derived epithelial cells were differentiating along the mesodermal lineage as opposed to ectodermal and endodermal. EGM2-MV is a proprietary microvascular endothelial media containing bFGF, VEGF, EGF, R³-IGF-1 [25] and FBS. These growth factors alone and in combination have been shown to play roles in mesoderm development [44-46], vascular development and vessel component differentiation [36,43,47] and EMT [48-55]. To our knowledge, there is no evidence in the literature for these growth factors inducing BMP4 expression in early development. It is possible that the removal of bFGF supplemented CM and the switch to EGM2-MV induced differentiation leading to increased BMP4 transcription. This induction combined with the exogenous growth factors may have preferentially caused the formation of mesoderm-like lineage.

Upon subculturing the cells, they began to undergo epithelial to mesenchymal (EMT) and take on mesenchymal phenotype. EMT is a critical process during development and cancer metastasis (Reviewed in [5]). Disruption of the intercellular connections mediated by E-cadherin is a signature event in EMT [56-58]. In our model, more than 80% of the epithelial cells expressed E-cadherin by flow cytometry. As the cells underwent apparent EMT, there was a decrease in E-cadherin, though its expression varied. This may be similar to the report by Boyer, et al, [59] where as NBT-II bladder carcinoma cells undergo EMT, E-cadherin cell-cell adhesions are disrupted and the protein is redistributed about the cell surface without a concomitant reduction in total protein. Of potential significance in EMT process described here is the up-regulation of GATA4 in the latter stages of the hESC to epithelial differentiation. GATA4 has been shown to play a critical role in cardiac and coronary development [8,60]. The coronary vasculature is fashioned by the epicardial epithelium undergoing EMT and differentiating into endothelial, smooth muscle and fibroblasts which assemble into vessels. It is possible the increased expression of GATA4 plays a role in the derived epithelial layer's transition to the mesenchymal phenotype.

The marker expression (positive: CD73, CD90, CD105, CD166; negative: CD31, CD34, CD45, CD133, CD146) and phenotype suggested the derived cells could be a type of mesenchymal progenitor cell. MSC-like cells have been derived from hESC [19-22]. These protocols have utilized co-culture of hESC on OP9 [19], manual isolation of differentiating cells at the edge of the hESC colonies [20,22] and subculture of sorted cells [19,21]. The protocol presented here is independent of feeders, manual selection or sorting for derivation of the mesenchymal cell lines. Another major difference with the aforementioned studies is the initial stem cell to epithelial formation. The present invention allows the hESC to develop a confluent monolayer that undergoes differentiation to the epithelial phenotype and it is when passaging resumes that the derived cells change phenotype. It is possible that once hESC initiate differentiation, if they are passaged at earlier stages or more frequently, they will bypass the epithelial state and directly become mesenchymal that can be selected as others have demonstrated (22).

To assess the functional capabilities of the derived cells, common protocols were used to test the hES-MC ability to differentiate along the three MSC lineages, osteogenic, chondrogenic and adipogenic [16,17]. Under the current culture conditions we were able to derive osteogenic and chondrogenic, but not adipogenic cells. It is well known that the ability of MSC to produce all three lineages is dependent upon culture conditions and as yet unknown factors in FBS [61]. One possibility in the lack of adipogenesis by the hES-MC is due to the medium they were cultured in since it is not commonly used for MSC maintenance and differentiation. Typical MSC medium uses FBS qualified for maintaining the MSC tri-lineage capacity without additional growth factors. In contrast, EGM2-MV is formulated for proliferation of mature microvascular endothelial cells with relatively low concentrations of bFGF, VEGF, EGF, R³-IGF-1 and in all likelihood, non-qualified FBS. These low levels of several growth factors may facilitate the formation of the mesoderm oriented epithelium and perhaps the EMT, but may limit the mesenchymal cells ability to become multiple lineages. The ability to produce osteogenic and chondrogenic, but not adipogenic cells fits in the differentiation hierarchy model proposed by Muraglia and colleagues [62]. They suggest MSC tri-potential indicates the earliest progenitor that upon maturation first looses its adipogenic capacity but can still produce osteo- and chondrogenic cells. As the MSC matures it next looses the chondrogenic function while retaining osteogenesis. Another possibility is the hES-MC are fibroblasts which contain some cells with MSC ability, as suggested by Sudo and colleagues [63].

Being a mesenchymal cell, the hES-MC were tested for their ability to remodel and contract a floating collagen I lattice. The experiments showed they have an equal or greater capacity to contract this 3D structure than mature keloid fibroblasts. MSC have also shown the ability to contract collagen I lattice [39]. This suggests these cells can sense the stresses within their environment and remodel it as has been shown in other cell types [38]. Also tested was the influence of TGF-β1 to induce expression of αSMA. In all cases, TGF-β1 exposed cultures showed up-regulation of αSMA protein levels while PDGF-B did not induce αSMA expression. This agrees with the findings of Gong and Niklason [40] using bone marrow derived MSC. They suggest MSC may have the innate capacity to differentiate to smooth muscle cells. Another possibility is the exposure to TGF-β1 is inducing a phenotype change to myofibroblasts. Myofibroblasts are activated fibroblasts that express αSMA under conditions of exposure to TGF-β1 [64].

### CONCLUSIONS

Monolayer culture is advantageous for controlling directed differentiation, minimizing undesired cell types and production scale-up compared to embryoid body differentiation. A primary use of the embryoid body is in vitro simulation of early embryo developmental processes [2,65]. Because of the potential to produce cells from all three germ layers, it could be more difficult to avoid contamination from multiple cell types. Although at this point our protocol cannot ensure absolutely one cell type, a monolayer approach should allow greater control over differentiation and facilitate scale-up as we have demonstrated with production of neural progenitor cells [41,42]. The derived hES-MC were highly proliferative and could have potential as feeder layers for hESC culture, wound healing models/therapies and large scale production of genetically controllable MSC. One of the reasons embryonic stem cells have generated so much excitement is their potential as a cell source in therapeutic applications. The hES-MC presented here may prove to play a small part in significantly advancing the state of the art.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

### REFERENCES

1 Bradley A, Evans M, Kaufman M H et al. Formation of germ-line chimaeras from embryo-derived teratocarcinoma cell lines. Nature 1984; 309:255-256.
2 Kleinsmith L J and Pierce G B, Jr. Multipotentiality of Single Embryonal Carcinoma Cells. Cancer Res 1964; 24:1544-1551.
3 Martin G R and Evans M J Differentiation of clonal lines of teratocarcinoma cells: formation of embryoid bodies in vitro. Proc Natl Acad Sci USA 1975; 72:1441-1445.
4 Martin G R Isolation of a pluripotent cell line from early mouse embryos cultured in medium conditioned by teratocarcinoma stem cells. Proc Natl Acad Sci USA 1981; 78:7634-7638.
5 Lee J M, Dedhar S, Kalluri R et al. The epithelial-mesenchymal transition: new insights in signaling, development, and disease. J Cell Biol 2006; 172:973-981.
6 Ohta S, Suzuki K, Tachibana K et al. Cessation of gastrulation is mediated by suppression of epithelial-mesenchymal transition at the ventral ectodermal ridge. Development 2007; 134:4315-4324.
7 Taneyhill L A, Coles E G, Bronner-Fraser M Snail2 directly represses cadherin6B during epithelial-to-mesenchymal transitions of the neural crest. Development 2007; 134:1481-1490.
8 Tevosian S G, Deconinck A E, Tanaka M et al. FOG-2, a cofactor for GATA transcription factors, is essential for heart morphogenesis and development of coronary vessels from epicardium. Cell 2000; 101:729-739.
9 Rivera-Feliciano J and Tabin C J Bmp2 instructs cardiac progenitors to form the heart-valve-inducing field. Dev Biol 2006; 295:580-588.
10 Xue C, Plieth D, Venkov C et al. The gatekeeper effect of epithelial-mesenchymal transition regulates the frequency of breast cancer metastasis. Cancer Res 2003; 63:3386-3394.
11 Graham T R, Zhau H E, Odero-Marah V A et al. Insulin-like growth factor-1-dependent up-regulation of ZEB1 drives epithelial-to-mesenchymal transition in human prostate cancer cells. Cancer Res 2008; 68:2479-2488.
12 Behr R, Heneweer C, Viebahn C et al. Epithelial-mesenchymal transition in colonies of rhesus monkey embryonic stem cells: a model for processes involved in gastrulation. Stem Cells 2005; 23:805-816.
13 Ullmann U, In't V P, Gilles C et al. Epithelial-mesenchymal transition process in human embryonic stem cells cultured in feeder-free conditions. Mol Hum Reprod 2007; 13:21-32.
14 Eastham A M, Spencer H, Soncin F et al. Epithelial-mesenchymal transition events during human embryonic stem cell differentiation. Cancer Res 2007; 67:11254-11262.
15 Thomson J A, Itskovitz-Eldor J, Shapiro S S et al. Embryonic stem cell lines derived from human blastocysts. Science 1998; 282:1145-1147.
16 Pittenger M F, Mackay A M, Beck S C et al. Multilineage potential of adult human mesenchymal stem cells. Science 1999; 284:143-147.
17 Bosch P, Pratt S L, Stice S L Isolation, characterization, gene modification, and nuclear reprogramming of porcine mesenchymal stem cells. Biol Reprod 2006; 74:46-57.
18 Bianchi G, Banfi A, Mastrogiacomo M et al. Ex vivo enrichment of mesenchymal cell progenitors by fibroblast growth factor 2. Exp Cell Res 2003; 287:98-105.
19 Barberi T, Willis L M, Socci N D et al. Derivation of multipotent mesenchymal precursors from human embryonic stem cells. PLoS Med 2005; 2:e161.
20 Olivier E N, Rybicki A C, Bouhassira E E Differentiation of human embryonic stem cells into bipotent mesenchymal stem cells. Stem Cells 2006; 24:1914-1922.
21 Lian Q, Lye E, Suan Y K et al. Derivation of clinically compliant MSCs from CD 105+. Stem Cells 2007; 25:425-436.
22 Trivedi P and Hematti P Derivation and immunological characterization of mesenchymal stromal cells from human embryonic stem cells. Exp Hematol 2008; 36:350-359.
23 Mitalipova M, Calhoun J, Shin S et al. Human embryonic stem cell lines derived from discarded embryos. Stem Cells 2003; 21:521-526.
24 Xu C, Inokuma M S, Denham J et al. Feeder-free growth of undifferentiated human embryonic stem cells. Nat Biotechnol 2001; 19:971-974.
25 Francis G L, Ross M, Ballard F J et al. Novel recombinant fusion protein analogues of insulin-like growth factor (IGF)-I indicate the relative importance of IGF-binding protein and receptor binding for enhanced biological potency. J Mol Endocrinol 1992; 8:213-223.
26 Boyd N L, Dhara S K, Rekaya R et al. BMP4 promotes formation of primitive vascular networks in human embryonic stem cell-derived embryoid bodies. Exp Biol Med (Maywood) 2007; 232:833-843.
27 Livak K J and Schmittgen T D Analysis of relative gene expression data using real-time quantitative PCR and the 2(-Delta Delta C(T)) Method. Methods 2001; 25:402-408.
28 Bosch P, Fouletier-Dilling C, Olmsted-Davis E A et al. Efficient adenoviral-mediated gene delivery into porcine mesenchymal stem cells. Mol Reprod Dev 2006; 73:1393-1403.
29 Ahrens P B, Solursh M, Reiter R S Stage-related capacity for limb chondrogenesis in cell culture. Dev Biol 1977; 60:69-82.
30 Pilcher B K, Kim D W, Carney D H et al. Thrombin stimulates fibroblast-mediated collagen lattice contraction by its proteolytically activated receptor. Exp Cell Res 1994; 211:368-373.
31 Kaufman D S, Lewis R L, Hanson E T et al. Functional endothelial cells derived from rhesus monkey embryonic stem cells. Blood 2004; 103:1325-1332.
32 Pimanda J E, Donaldson U, de Bruijn M F et al. The SCL transcriptional network and BMP signaling pathway interact to regulate RUNX1 activity. Proc Natl Acad Sci USA 2007; 104:840-845.
33 Rojas A, De Val S, Heidt A B et al. Gata4 expression in lateral mesoderm is downstream of BMP4 and is activated directly by Forkhead and GATA transcription factors through a distal enhancer element. Development 2005; 132:3405-3417.
34 Adewumi O, Aflatoonian B, Ahrlund-Richter L et al. Characterization of human embryonic stem cell lines by the International Stem Cell Initiative. Nat. Biotechnol 2007; 25:803-816.
35 Shin S, Dalton S, Stice S L Human motor neuron differentiation from human embryonic stem cells. Stem Cells Dev 2005; 14:266-269.
36 Yamashita J, Itoh H, Hirashima M et al. Flk1-positive cells derived from embryonic stem cells serve as vascular progenitors. Nature 2000; 408:92-96.
37 Lev R and Spicer S S Specific Staining of Sulphate Groups with Alcian Blue at Low Ph. J Histochem Cytochem 1964; 12:309.
38 Bell E, Ivarsson B, Merrill C Production of a tissue-like structure by contraction of collagen lattices by human fibroblasts of different proliferative potential in vitro. Proc Natl Acad Sci USA 1979; 76:1274-1278.
39 Badillo A T, Redden R A, Zhang L et al. Treatment of diabetic wounds with fetal murine mesenchymal stromal cells enhances wound closure. Cell Tissue Res 2007; 329:301-311.
40 Gong Z and Niklason L E Small-diameter human vessel wall engineered from bone marrow-derived mesenchymal stem cells (hMSCs). FASEB J 2008
41 Shin S, Mitalipova M, Noggle S et al. Long-term proliferation of human embryonic stem cell-derived neuroepithelial cells using defined adherent culture conditions. Stem Cells 2006; 24:125-138.
42 Dhara S K, Hasneen K, Machacek D W et al. Human neural progenitor cells derived from embryonic stem cells in feeder-free cultures. Differentiation 2008
43 Levenberg S, Golub J S, Amit M et al. Endothelial cells derived from human embryonic stem cells. Proc Natl Acad Sci USA 2002; 99:4391-4396.
44 Slack J M, Darlington B G, Heath J K et al. Mesoderm induction in early Xenopus embryos by heparin-binding growth factors. Nature 1987; 326:197-200.
45 Gillespie L L, Paterno G D, Mahadevan L C et al. Intracellular signalling pathways involved in mesoderm induction by FGF. Mech Dev 1992; 38:99-107.
46 Kimelman D and Kirschner M Synergistic induction of mesoderm by FGF and TGF-beta and the identification of an mRNA coding for FGF in the early Xenopus embryo. Cell 1987; 51:869-877.
47 Shalaby F, Rossant J, Yamaguchi T P et al. Failure of blood-island formation and vasculogenesis in Flk-1-deficient mice. Nature 1995; 376:62-66.
48 Strutz F, Zeisberg M, Ziyadeh F N et al. Role of basic fibroblast growth factor-2 in epithelial-mesenchymal transformation. Kidney Int 2002; 61:1714-1728.
49 Tanaka T, Saika S, Ohnishi Y et al. Fibroblast growth factor 2: roles of regulation of lens cell proliferation and epithelial-mesenchymal transition in response to injury. Mol V is 2004; 10:462-467.
50 Dor Y, Camenisch T D, Itin A et al. A novel role for VEGF in endocardial cushion formation and its potential contribution to congenital heart defects. Development 2001; 128:1531-1538.
51 Rodgers L S, Lalani S, Hardy K M et al. Depolymerized hyaluronan induces vascular endothelial growth factor, a negative regulator of developmental epithelial-to-mesenchymal transformation. Circ Res 2006; 99:583-589.
52 Zhang Y, Pan Q, Zhong H et al. Inhibition of CCN6 (WISP3) expression promotes neoplastic progression and enhances the effects of insulin-like growth factor-1 on breast epithelial cells. Breast Cancer Res 2005; 7:R1080-R1089.
53 Irie H Y, Pearline R V, Grueneberg D et al. Distinct roles of Akt1 and Akt2 in regulating cell migration and epithelial-mesenchymal transition. J Cell Biol 2005; 171:1023-1034.
54 Lo H W, Hsu S C, Xia W et al. Epidermal growth factor receptor cooperates with signal transducer and activator of transcription 3 to induce epithelial-mesenchymal transition in cancer cells via up-regulation of TWIST gene expression. Cancer Res 2007; 67:9066-9076.
55 Tian Y C, Chen Y C, Chang C T et al. Epidermal growth factor and transforming growth factor-beta1 enhance HK-2 cell migration through a synergistic increase of matrix metalloproteinase and sustained activation of ERIC signaling pathway. Exp Cell Res 2007; 313:2367-2377.
56 Perl A K, Wilgenbus P, Dahl U et al. A causal role for E-cadherin in the transition from adenoma to carcinoma. Nature 1998; 392:190-193.
57 Cano A, Perez-Moreno M A, Rodrigo I et al. The transcription factor snail controls epithelial-mesenchymal transitions by repressing E-cadherin expression. Nat Cell Biol 2000; 2:76-83.
58 Batlle E, Sancho E, Franci C et al. The transcription factor snail is a repressor of E-cadherin gene expression in epithelial tumour cells. Nat Cell Biol 2000; 2:84-89.
59 Boyer B, Dufour S, Thiery J P E-cadherin expression during the acidic FGF-induced dispersion of a rat bladder carcinoma cell line. Exp Cell Res 1992; 201:347-357.
60 Crispino J D, Lodish M B, Thurberg B L et al. Proper coronary vascular development and heart morphogenesis depend on interaction of GATA-4 with FOG cofactors. Genes Dev 2001; 15:839-844.
61 Caterson E J, Nesti L J, Danielson K G et al. Human marrow-derived mesenchymal progenitor cells: isolation, culture expansion, and analysis of differentiation. Mol Biotechnol 2002; 20:245-256.
62 Muraglia A, Cancedda R, Quarto R Clonal mesenchymal progenitors from human bone marrow differentiate in vitro according to a hierarchical model. J Cell Sci 2000; 113 (Pt 7):1161-1166.
63 Sudo K, Kanno M, Miharada K et al. Mesenchymal progenitors able to differentiate into osteogenic, chondrogenic, and/or adipogenic cells in vitro are present in most primary fibroblast-like cell populations. Stem Cells 2007; 25:1610-1617.
64 Vaughan M B, Howard E W, Tomasek J J Transforming growth factor-beta1 promotes the morphological and functional differentiation of the myofibroblast. Exp Cell Res 2000; 257:180-189.
65 Doetschman T C, Eistetter H, Katz M et al. The in vitro development of blastocyst-derived embryonic stem cell lines: formation of visceral yolk sac, blood islands and myocardium. J Embryol Exp Morphol 1985; 87:27-45.

1. A method of producing mesenchymal-like stem cells (MSCs) from pluripotent stem cells (PSCs) comprising:
i. Exposing PSCs in culture to a stem cell conditioning medium, optionally on a substrate or differentiation protein, to make the cells confluent;
ii. Exposing the confluent PSCs to a differentiation medium, optionally on a substrate or differentiation protein, wherein said medium comprises effective amounts of fibroblast growth factor, vascular endothelial growth factor (VEGF) and insulin-like growth factor (IGF), and optionally, epidermal growth factor (EGF), hydrocortisone or a mixture of epidermal growth factor and hydrocortisone for a period of time effective to produce a population of pluripotent stem cell derived epithelial cells;
iii. Optionally isolating said stem cell derived epithelial cells;
iv. Exposing the stem cell derived epithelial cells from step ii or step iii to a differentiation medium, optionally on a substrate or differentiation protein, comprising effective amounts of fibroblast growth factor, especially basic fibroblast growth factor (bFGF), vascular endothelial growth factor (VEGF) and insulin-like growth factor (IGF), especially IGF-1 (including recombinant versions of IGF-1 such as R³-IGF-1 and optionally, epidermal growth factor (EGF) and/or hydrocortisone for a period effective to differentiate said stem cell derived epithelial cells (preferably, hESC-EC) to stem cell derived mesenchymal cells (preferably hESC-MC); and
v. Optionally, isolating said mesenchymal cells.

2. The method according to claim 1 wherein said mesenchymal cells obtained from step iv or step v are further differentiated into bone, cartilage or smooth muscle tissue by exposing the mesenchymal cells to a differentiation medium for a period of at least about 24 hours.

3. The method according to claim 1 wherein said PSCs are human embryonic stem cells (hESCs), said epithelial cells are human embryonic stem cell derived epithelial cells (hESC-ECs) and said mesenchymal cells are human embryonic stem cell derived mesenchymal cells (hESC-MCs).

4. The method according to claim 1 wherein said epithelial cells are grown to a uniform sheet.

5. The method according to claim 1 wherein said fibroblast growth factor is basic fibroblast growth factor (bFGF).

6. The method according to claim 1 wherein said insulin-like growth factor is IGF-1.

7. The method according to claim 4 wherein said IGF-1 is recombinant R³-IGF-1.

8. The method according to claim 1 wherein fibroblast growth factor is basic fibroblast growth factor (bFGF), said insulin-like growth factor is IGF-1 and said differentiation medium further comprises epidermal growth factor (EGF) and hydrocortisone.

9. The method according to claim 1 wherein said pluripotent stem cells (step ii) are exposed to said differentiation medium for a period of between about 1 and 20 days to produce stem cell derived epithelial cells.

10. The method according to claim 1 wherein said pluripotent stem cell derived epithelial cells are exposed to differentiation medium for a period ranging from about 1 and 15 days.

11. The method according to claim 8 wherein said epithelial cells are exposed to said differentiation medium for a period ranging from about 5 and 10 days to produce mesenchymal cells.

12. The method according to claim 2 wherein said smooth muscle cells are vascular cells or cardiovascular cells.

13. The method according to claim 1 wherein said substrate or differentiation medium is selected from the group consisting of laminin, tenascin, thrombospondin, collagen, fibronectin, vibronectin, polylysine, polyornithine and mixtures thereof.

14. The method according to claim 1 wherein said substrate or differentiation medium is laminin.

15. The method according to claim 1 wherein said epithelial cells are differentiated on a substrate or differentiation protein and said mesenchymal cells are isolated solely by passaging and collecting said cells without a further isolation step.

16. A method of producing human embryonic stem cell derived mesenchymal cells (hESC-MCs) from human embryonic stem cells comprising:
i. Exposing human embryonic stem cells (hESCs) to a differentiation medium, optionally on a substrate or differentiation protein, wherein said medium comprises effective amounts of fibroblast growth factor, vascular endothelial growth factor (VEGF) and insulin-like growth factor (IGF), and optionally, epidermal growth factor (EGF), hydrocortisone or a mixture of epidermal growth factor and hydrocortisone for a period of time effective to produce a population of human embryonic stem cell derived epithelial cells (hESC-ECs);
ii. Optionally isolating said stem cell derived epithelial cells;
iii. Exposing the stem cell derived epithelial cells from step ii or step iii to a differentiation medium, optionally on a substrate or differentiation protein, comprising effective amounts of fibroblast growth factor, especially basic fibroblast growth factor (bFGF), vascular endothelial growth factor (VEGF) and insulin-like growth factor (IGF), especially IGF-1 (including recombinant versions of IGF-1 such as R³-IGF-1 and optionally, epidermal growth factor (EGF) and/or hydrocortisone for a period effective to differentiate said stem cell derived epithelial cells to human embryonic stem cell derived mesenchymal cells (hESC-MCs); and
iv. Optionally, isolating said mesenchymal cells.

17. The method according to claim 16 wherein said mesenchymal cells obtained from step iv or step v are further differentiated into bone, cartilage or smooth muscle tissue by exposing the hESC-MCs to a differentiation medium for a period of at least about 24 hours.

18. The method according to claims 16 wherein said epithelial cells are grown to a uniform sheet.

19. The method according to claim 16 wherein said fibroblast growth factor is basic fibroblast growth factor (bFGF).

20. The method according to claim 16 wherein said insulin-like growth factor is IGF-1.

21. The method according to claim 20 wherein said IGF-1 is recombinant R³-IGF-1.

22. The method according to claim 16 wherein said fibroblast growth factor is basic fibroblast growth factor (bFGF), said insulin-like growth factor is IGF-1 and said differentiation medium further comprises epidermal growth factor (EGF) and hydrocortisone.

23. The method according to claim 16 wherein said human embryonic stem cells (step i) are exposed to said differentiation medium for a period of between about 1 and 20 days to produce stem cell derived epithelial cells.

24. The method according to claim 16 wherein said human embryonic stem cells are exposed to differentiation medium for a period ranging from about 5 and 20 days to produce human embryonic stem cell derived epithelial cells (hESC-ECs).

25. The method according to claim 16 wherein said epithelial cells are exposed to said differentiation medium for a period ranging from about 5 and 10 days to produce mesenchymal cells.

26. The method according to claim 17 wherein said smooth muscle cells are vascular cells or cardiovascular cells.

27. A method of producing human embryonic stem cell derived epithelial cells (hESC-ECs) from human embryonic stem cells comprising:
i. Exposing human embryonic stem cells (hESCs) to a differentiation medium, optionally on a substrate or differentiation protein, wherein said medium comprises effective amounts of fibroblast growth factor, vascular endothelial growth factor (VEGF) and insulin-like growth factor (IGF), and optionally, epidermal growth factor (EGF), hydrocortisone or a mixture of epidermal growth factor and hydrocortisone for a period of time effective to produce a population of human embryonic stem cell derived epithelial cells (hESC-ECs);
ii. Optionally isolating said stem cell derived epithelial cells.

28. The method according to claim 27 wherein said epithelial cells obtained from step i or step ii are exposed to a differentiation medium, optionally on a substrate or differentiation protein, comprising effective amounts of fibroblast growth factor, especially basic fibroblast growth factor (bFGF), vascular endothelial growth factor (VEGF) and insulin-like growth factor (IGF), especially IGF-1 (including recombinant versions of IGF-1 such as R³-IGF-1 and optionally, epidermal growth factor (EGF) and/or hydrocortisone for a period effective to differentiate said stem cell derived epithelial cells to human embryonic stem cell derived mesenchymal cells (hESC-MCs); and
Optionally, isolating said mesenchymal cells.

29. The method according to claim 28 wherein said mesenchymal cells are further differentiated into bone, cartilage or smooth muscle tissue by exposing the hESC-MCs to a differentiation medium for a period of at least about 24 hours.

30. The method according to claim 27 wherein said epithelial cells are grown to a uniform sheet.

31. The method according to claim 27 wherein said fibroblast growth factor is basic fibroblast growth factor (bFGF).

32. The method according to claim 27 wherein said insulin-like growth factor is IGF-1.

33. The method according to claim 31 wherein said IGF-1 is recombinant R³-IGF-1.

34. The method according to claim 27 wherein said fibroblast growth factor is basic fibroblast growth factor (bFGF), said insulin-like growth factor is IGF-1 and said differentiation medium further comprises epidermal growth factor (EGF) and hydrocortisone.

35. The method according to claim 27 wherein said human embryonic stem cells (step i) are exposed to said differentiation medium for a period of between about 1 and 20 days to produce said stem cell derived epithelial cells.

36. The method according to claim 27 wherein said human embryonic stem cells are exposed to differentiation medium for a period ranging from about 5 and 20 days to produce human embryonic stem cell derived epithelial cells (hESC-ECs).

37. The method according to claim 27 wherein said epithelial cells are exposed to said differentiation medium for a period ranging from about 5 and 10 days to produce mesenchymal cells.

38. The method according to claim 29 wherein said smooth muscle cells are vascular cells or cardiovascular cells.

39. A method of producing human embryonic stem cell derived mesenchymal cells (hESC-MCs) directly from human embryonic stem cells comprising:
i. Exposing human embryonic stem cells (hESCs) to a differentiation medium, optionally on a substrate or differentiation protein, wherein said medium comprises effective amounts of fibroblast growth factor, vascular endothelial growth factor (VEGF) and insulin-like growth factor (IGF), and optionally, epidermal growth factor (EGF), hydrocortisone or a mixture of epidermal growth factor and hydrocortisone for a period of about 10 to 20 days;
ii. Passaging said cells obtained in step i and exposing said passaged cells to a differentiation medium, optionally on a substrate or differentiation protein, comprising effective amounts of fibroblast growth factor, especially basic fibroblast growth factor (bFGF), vascular endothelial growth factor (VEGF) and insulin-like growth factor (IGF), especially IGF-1 (including recombinant versions of IGF-1 such as R³-IGF-1 and optionally, epidermal growth factor (EGF) and/or hydrocortisone for a period effective to differentiate said stem cell derived epithelial cells to human embryonic stem cell derived mesenchymal cells (hESC-MCs); and
Optionally, isolating said mesenchymal cells.

40. The method according to claim 39 wherein said mesenchymal cells are further differentiated into bone, cartilage or smooth muscle tissue by exposing the hESC-MCs to a differentiation medium for a period of at least about 24 hours.

41. The method according to claim 39 wherein said fibroblast growth factor is basic fibroblast growth factor (bFGF).

42. The method according to claim 39 wherein said insulin-like growth factor is IGF-1.

43. The method according to claim 41 wherein said IGF-1 is recombinant R³-IGF-1.

44. The method according to claim 39 wherein said fibroblast growth factor is basic fibroblast growth factor (bFGF), said insulin-like growth factor is IGF-1 and said differentiation medium further comprises epidermal growth factor (EGF) and hydrocortisone.

45. The method according to claim 39 wherein said smooth muscle cells are vascular cells or cardiovascular cells.

46. An epithelial cell produced according to the method of any of claims 1, 16 or 27.

47. A mesenchymal cell produced according to the method of any of claims 1, 16, 28 and 39.

48. A human embryonic stem cell derived epithelial cell exhibiting at least 4 of the following characteristics:
They can be cultured as a stable cell population;
Cells appear in an epithelial layer and exhibit mesodermal gene expression patterns;
Cells are positive for the following markers: BMP4, RUNX1, GATA4;
Cells can be produced from a range of hESC lines including BG01, BG02, WA09;
Cells express E-cadherin (E-cadherin⁺);
Cells express low levels of CD90 (CD90^{low});
Can be isolated, frozen and cryogenically preserved by standard methods;
Can be recovered after cryogenic storage, recovered and differentiated;
Can be passaged with high plating efficiency (greater than 50% plating efficiency-50% of cells passaged successfully seed down and survive);
hESC-ECs retain a normal karyotype during passaging;
Have multipotent differentiation capacity (epithelial and mesenchymal-like (hESC-MC);
They may be cultured as a monolayer;
They require no selection or isolation techniques including but not limited to genetic markers or phenotypic characterization for a MSC phenotype; and
They may be genetically manipulated.

49. The human embryonic stem cell derived epithelial cell according to claim 48 exhibiting at least 10 of said characteristics.

50. The human embryonic stem cell derived epithelial cell according to claim 48 exhibiting all 14 of said characteristics.

51. A human embryonic stem cell derived mesenchymal cell exhibiting at least 4 of the following characteristics:
They can be cultured for at least 10 passages as a stable cell population;
Cells appear mesenchymal and have numerous mesenchymal stem cell markers including CD73, CD90, CD105 and CD166;
can be produced from a range of hESC lines including BG01, BG02, WA09;
hESC-MCs can be frozen and cryogenically preserved by standard methods;
hESC-MCs can be recovered after cryogenic storage, recovered and differentiated;
hESC-MCs can be passaged with high plating efficiency (greater than 50% plating efficiency-50% of cells passaged successfully seed down and survive);
do not exhibit the hematopoietic markers CD34, CD45 and CD133 on their cell surface;
do not express endothelial markers CD31 and CD146;
hESC-MCs are E-cadherin negative;
hESC-MCs retain a normal karyotype during passaging;
hESC-MCs exhibit a mesenchymal phenotype;
hESC-MCs are able to remodel and contract collagen I lattice constructs to an equivalent degree as keloid fibroblast control cells;
TGF-β1, but not PDGF-B induces expression of αSMA;
have multipotent differentiation capacity (including osteogenic and chondrogenic);
do not exhibit lipogenic differentiation capacity when exposed to standard lipogenic conditions (high glucose MEM Alpha, supplemented with ITS+1, sodium pyruvate (10 mM), methyl isobutylxanthine (0.5 mM) and dexamethasone (1 μM);
The may be cultured as a monolayer;
No selection or isolation techniques are required including but not limited to genetic; markers or phenotypic characterization for a MSC phenotype;
They pass through a early mesodermal phenotype and epithelial phenotype prior to forming a MSC; and
They can be genetically manipulated.

52. The human embryonic stem cell derived mesenchymal cell according to claim 51 exhibiting at least 10 of said characteristics.

53. The human embryonic stem cell derived mesenchymal cell according to claim 51 exhibiting at least 15 of said characteristics.

54. The human embryonic stem cell derived mesenchymal cell according to claim 51 exhibiting all 19 of said characteristics.

55. An epithelial cell according to claim 45 wherein said cell is cryopreserved.

56. A mesenchymal cell according to claim 46 wherein said cell is cryopreserved.
